SEQUENCE LISTING (20 sequences)

SEQ ID NO: 1 - protein, 169 aa, Artificial Sequence
  Description of Artificial Sequence Synthetic polypeptide
AAANSPFADPNTAFAGFDENKVTVLTIVTEHTDLTMGWSSIILFLLATATGVHSQVQLQQPGAELVRPGASVKLSCKASGYSFTSDWMNWVKQRPGQGLEWIGIIHPSDSETKINQYFKDKATLTIDKSSSTAYMQLSSPTSEDSAVYYCVRWVGYHAQVWGQGTTLTV

SEQ ID NO: 2 - protein, 140 aa, Artificial Sequence
  Description of Artificial Sequence Synthetic polypeptide
ISQEQKFKDKMDFQVQIFSFLLISFSVIMSRGQIVLTQSPAIMSVSPGEKVTLTCNASSSVSSSYLYWYRQKPGSSPNLWIYSTSNLASGVPARFSGSGSGTSYSLTISSMEAEDVASYFCHQWSSYPLTFGAGTKLELK

SEQ ID NO: 3 - DNA, 507 bp, Artificial Sequence
  Description of Artificial Sequence Synthetic polynucleotide
gcggccgcgaattcgcccttcgcggatccgaacactgcgtttgctggctttgatgaaaataaggtcactgttctcactatagttactgagcacacagacctcaccatgggatggagctctatcatcctcttcttgttagcaacagctacaggtgtccactcccaggtccaactgcagcagcctggggctgagctggtgaggcctggagcttcagtgaagctgtcctgcaaggcttctggctactccttcaccagcgactggatgaactgggtgaaacagaggcctggacagggccttgagtggattggcataattcatccttccgacagtgaaactaaaataaatcaatatttcaaggacaaggccacattgactatagacaaatcctccagcacagcctacatgcaactcagcagcccgacatctgaggactctgcggtctattactgtgttagatgggtgggctaccacgctcaggtctggggccaaggcaccactctcaccgtc

SEQ ID NO: 4 - DNA, 420 bp, Artificial Sequence
  Description of Artificial Sequence Synthetic polynucleotide
attagccaggaacaaaaattcaaagacaaaatggattttcaggtgcagattttcagcttcctgctaatcagtttctcagtcataatgtccagaggacaaattgttctcacccagtctccagcaatcatgtctgtatctccgggggagaaagtcaccttgacctgcaatgccagctcaagtgtaagttccagctacttatactggtatcggcagaaaccaggatcttcccccaacctctggatttatagcacatccaacctggcttctggagtccctgctcgcttcagtggcagtgggtctgggacctcttactctctcacaatcagcagcatggaggctgaagatgttgcctcttatttctgccatcagtggagtagttacccgctcacgttcggtgctgggaccaagctggagctgaag

SEQ ID NO: 5 - protein, 10 aa, Artificial Sequence
  Description of Artificial Sequence Synthetic peptide
GYSFTSDWMN

SEQ ID NO: 6 - protein, 17 aa, Artificial Sequence
  Description of Artificial Sequence Synthetic peptide
IIHPSDSETKINQYFKD

SEQ ID NO: 7 - protein, 8 aa, Artificial Sequence
  Description of Artificial Sequence Synthetic peptide
WVGYHAQV

SEQ ID NO: 8 - protein, 12 aa, Artificial Sequence
  Description of Artificial Sequence Synthetic peptide
NASSSVSSSYLY

SEQ ID NO: 9 - protein, 7 aa, Artificial Sequence
  Description of Artificial Sequence Synthetic peptide
STSNLAS

SEQ ID NO: 10 - protein, 9 aa, Artificial Sequence
  Description of Artificial Sequence Synthetic peptide
HQWSSYPLT

SEQ ID NO: 11 - DNA, 36 bp, Artificial Sequence
  Description of Artificial Sequence Synthetic primer
cgcggatcccaatggcacaagtcattaatacaaaca

SEQ ID NO: 12 - DNA, 37 bp, Artificial Sequence
  Description of Artificial Sequence Synthetic primer
tccgctcgagttaaatagtttcaccgtcgttggcacc

SEQ ID NO: 13 - protein, 365 aa, Proteus mirabilis
MAQVINTNYLSLVTQNNLNKSQGTLGSAIERLSSGLRINSAKDDAAGQAIANRFTSNVNGLTQASRNANDGISIAQTTEGALNEINNNLQRIRELTVQAKNGTNSNSDITSIQNEVKNVLDEINRISEQTQFNGVKVLSGEKSEMVIQVGTNDNETIKFNLDKVDNDTLGVASDKLFDTKTEKKGVTAAGAGVTDAKKINAAATLDMMVSLVKEFNLDGKPVTDKFIVTKGGKDYVATKSDFELDATGTKLGLKASATTEFKVDAGKDVKTLNVKDDALATLDKAINTIDESRSKLGAIQNRFESTINNLNNTVNNLSASRSRILDADYATEVSNMSRGQILQQAGTSVLAQANQVPQTVLSLLR

SEQ ID NO: 14 - protein, 387 aa, Pseudomonas aeruginosa
MALTVNTNIASLNTQRNLNNSSASLNTSLQRLSTGSRINSAKDDAAGLQIANRLTSQVNGLNVATKNANDGISLAQTAEGALQQSTNILQRMRDLSLQSANGSNSDSERTALNGEVKQLQKELDRISNTTTFGGRKLLDGSFGVASFQVGSAANEIISVGIDEMSAESLNGTYFKADGGGAVTAATASGTVDIAIGITGGSAVNVKVDMKGNETAEQAAAKIAAAVNDANVGIGAFTDGAQISYVSKASADGTTSAVSGVAITDTGSTGAGTAAGTTTFTEANDTVAKIDISTAKGAQSAVLVIDEAIKQIDAQRADLGAVQNRFDNTINNLKNIGENVSAARGRIEDTDFAAETANLTKNQVLQQAGTAILAQANQLPQSVLSLLR

SEQ ID NO: 15 - protein, 585 aa, Escherichia coli
MAQVINTNSLSLITQNNLNKNQSALSSSIERLSSGLRINSAKDDAAGQAIANRFTSNIKGLTQAARNANDGISVAQTTEGALSEINNNLQRIRELTVQATTGTNSDSDLDSIQDEIKSRLDEIDRVSGQTQFNGVNVLAKDGSMKIQVGANDGETITIDLKKIDSDTLGLNGFNVNGKGTITNKAATVSDLTSAGAKLNTTTGLYDLKTENTLLTTDAAFDKLGNGDKVTVGGVDYTYNAKSGDFTTTKSTAGTGVDAAAQAADSASKRDALAATLHADVGKSVNGSYTTKDGTVSFETDSAGNITIGGSQAYVDDAGNLTTNNAGSAAKADMKALLKAASEGSDGASLTFNGTEYTIAKATPATTTPVAPLIPGGITYQATVSKDVVLSETKAAAATSSITFNSGVLSKTIGFTAGESSDAAKSYVDDKGGITNVADYTVSYSVNKDNGSVTVAGYASATDTNKDYAPAIGTAVNVNSAGKITTETTSAGSATTNPLAALDDAISSIDKFRSSLGAIQNRLDSAVTNLNNTTTNLSEAQSRIQDADYATEVSNMSKAQIIQQAGNSVLAKANQVPQQVLSLLQG

SEQ ID NO: 16 - protein, 348 aa, Serratia marcescens
MAQVINTNSLSLMAQNNLNKSQSSLGTAIERLSSGLRINSAKDDAAGQAISDRFTANIKGLTQASRNANDGISLAQTTEGALNEVNDNLQNIRRLTVQAQNGSNSTSDLKSIQDEITQRMSEINRISEQTDFNGVKVLSSDQKLTIQVGANDGETIDIDLQGLTGFDVTENGTKIGSAIADKAMVKDDTGTDVAFDLGESFQTGGALEKATLVSGKTKDGKEGYYIQTTDAATGAKTYATAKIDDKGVVTKGADVTDVKDPLATLDKALAQVDGLRSSLGAVQNRFDSVISNLNSTVNNLSASQSRIQDADYATEVSNMSRAHILQQAGTSVLAQANQSTQNVLSLLR

SEQ ID NO: 17 - protein, 505 aa, Salmonella muenchen
MAQVINTNSLSLLTQNNLNKSQSALGTAIERLSSGLRINSAKDDAAGQAIANRFTANIKGLTQASRNANDGISIAQTTEGALNEINNNLQRVRELAVQSANGTNSQSDLDSIQAEITQRLNEIDRVSGQTQFNGVKVLAQDNTLTIQVGANDGETIDIDLKEISSKTLGLDKLNVQDAYTPKETAVTVDKTTYKNGTDTITAQSNTDIQTAIGGGATGVTGADIKFKDGQYYLDVKGGASAGVYKATYDETTKKVNIDTTDKTPLATAEATAIRGTATITHNQIAEVTKEGVDTTTVAAQLAAAGVTGADKDNTSLVKLSFEDKNGKVIDGGYAVKMGDDFYAATYDEKQVQLLLNNHYTDGAGVLQTGAVKFGGANGKSEVVTATVGKTYLASDLDKHNFRTGGELKEVNTDKTENPLQKIDAALAQVDTLRSDLGAVQNRFNSAITNLGNTVNNLSSARSRIEDSDYATEVSNMSRAQILQQAGTSVLAQANQVPQNVLSLLR

SEQ ID NO: 18 - protein, 490 aa, Salmonella typhimurium
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVNGQTQFSGVKVLAQDNTLTIQVGANDGETIDIDLKQINSQTLGLDTLNVQQKYKVSDTAATVTGYADTTIALDNSTFKASATGLGGTDEKIDGDLKFDDTTGKYYAKVTVTGGTGKDGYYEVSVDKTNGEVTLAAVTPATVTTATALSGKMYSANPDSDIAKAALTAAGVTGTASVVKMSYTDNNGKTIDGGLAVKVGDDYYSATQDKDGSISIDTTKYTADNGTSKTALNKLGGADGKTEVVTIDGKTYNASKAAGHDFKAEPELAEQAAKTTENPLQKIDAALAQVDTLRSDLGAVQNRFNSAITNLGNTVNNLSSARSRIEDSDYATEVSNMSRAQILQQAGTSVLAQANQVPQNVLSLLR

SEQ ID NO: 19 - DNA, 468 bp, Salmonella muenchen
aaggaaaagatcatggcacaagtcattaatacaaacagcctgtcgctgttgacccagaataacctgaacaaatcccagtccgctctgggcaccgctatcgagcgtctgtcttccggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacggtactaactcccagtctgaccttgactctatccaggctgaaatcacccagcgtctgaacgaaatcgaccgtgtatccggtcagactcagttcaacggcgtgaaagtcctggcgcaggacaacaccctgaccatccaggttggtgccaacgac

SEQ ID NO: 20 - protein, 23 aa, Salmonella muenchen
RLSSGLRINSAKDDAAGQAIANR

(19) United States
(12) Patent Application Publication
(10) Pub. No.: US 2010/0239583 A1
(43) Pub. Date: Sep. 23, 2010

(54) ANTIBODIES AGAINST FLAGELLIN AND USES THEREOF

(75) Inventors: Kanneganti MURTHY, Stoneham, MA (US); Andrew L. SALZMAN, Herzliya (IL)

Correspondence Address: LAHIVE & COCKFIELD, LLP;FLOOR 30, SUITE 3000, ONE POST OFFICE SQUARE, BOSTON, MA, 02109, US

(73) Assignee: INOTEK PHARMACEUTICALS CORPORATION, Beverly, MA (US)

(21) Appl. No.: 12/717,351

(22) Filed: Mar. 4, 2010

Related U.S. Application Data
(60) Provisional application No. 61/209,189, filed on Mar. 4, 2009.

(51) Int. Cl. A61K 39/395; C07K 16/12; C07K 16/46; C12N 5/16; C07H 21/04; C12N 15/13; A01N 63/02; A01P 1/00; A61P 31/04; A61P 31/08; A61P 31/06; A61P 13/02; A61P 7/00; G01N 33/53

(52) U.S. Cl. 424/136.1; 530/388.4; 530/389.5; 530/387.9; 530/387.3; 530/388.15; 435/340; 530/391.7; 424/150.1; 424/164.1; 424/178.1; 536/23.53; 435/320.1; 435/7.1

(57) ABSTRACT

The present invention provides a novel class of monoclonal antibodies which have a high affinity, broad spectrum neutralizing reactivity to flagellin from various Gram-negative bacteria including, but not limited to, E. coli, Salmonella, Serratia, Proteus, Enterobacter, Citrobacter, Campylobacter and Pseudomonas. The present invention further provides methods of treating infections and diseases using anti-flagellin antibodies in humans, other animals and birds.

### RELATED APPLICATION

This application claims the benefit of U.S. provisional application No. 61/209,189, filed Mar. 4, 2009, which is incorporated by reference herein in its entirety.

### GOVERNMENT FUNDING

This invention was made with government support under National Institutes of Health Grant #'s R01GM57407, R43AI48249, R29GM54773 and R01GM60699. The United States government has certain rights in the invention.

### BACKGROUND OF THE INVENTION

The human intestine is colonized by a large and diverse population of commensal bacteria and, on occasion, is exposed to potentially pathogenic bacteria. One particular subset of intestinal bacteria have flagella, which are whip-like organelles that attach to a rotatory motor embedded in the bacterial cell wall. Flagella provide bacteria with motility and enable these microbes to reach, adhere and eventually invade or colonize a particular niche in their host. An individual flagellum is composed of approximately 20,000 subunits of the monomeric protein flagellin. Due to physical constraints by its function, flagellin has a relatively conserved structure among widely diverse bacterial species (Steiner, T. S. Infect Immun. 2007 February; 75(2):545-52).

Flagellin is highly antigenic and is a major immunoglobulin target in a variety of infectious events (Sitaraman et al., Am J Physiol Gastrointest Liver Physiol. 2005 February; 288(2):G403-6). As such, it is a potent and direct activator of the innate immune system. From the perspective of the host, flagellin is a microbial-associated molecular pattern (MAMP) i.e., a microbial-associated determinant that can be perceived by the innate immune system, typically by pattern recognition receptors. Flagellin therefore serves as a danger signal across a wide variety of eukaryotes and is a potent inducer of inflammatory effector responses in the mammalian gut (Neish, A. S., Am J Physiol Gastrointest Liver Physiol. 2007 February; 292(2):G462-6).

Specifically, upon detection of miniscule levels of the monomeric protein flagellin, the mammalian germline encoded cell surface receptor Toll-like receptor 5 (TRL5) can directly promote a mucosal inflammatory response and trigger a massive induction of host gene expression designed to arm and protect the host against the invading microbe. The resulting inflammatory cascade triggered by flagellin can be profound, causing clinical manifestations and tissue damage (Gewirtz, A. T., Am J Physiol Gastrointest Liver Physiol. 2007 March; 292(3):G706-10).

Current treatments to offset the deleterious effects of an inflammatory cascade stimulated by the flagellin include anti-inflammatory drugs, immune system suppressors and other over-the-counter (OTC) drugs. These therapies, however, have clear drawbacks in that they are associated with undesirable side effects and are merely palliative in nature. Accordingly, improved agents and therapeutic treatments would be beneficial.

### SUMMARY OF THE INVENTION

The present invention provides antibodies that bind to flagellin and neutralize a broad spectrum of bacteria including, but not limited to, gram-negative bacteria, such as E. coli, Salmonella, Serratia, Proteus, Enterobacter, Citrobacter, Campylobacter and Pseudomonas. Accordingly, the antibodies of the present invention can be used to treat, prevent and diagnose a variety of bacterial diseases associated with flagellin, including both infectious and non-infectious diseases in humans, other animals and birds.

Antibodies of the invention generally are characterized as having one or more of the following properties: (i) neutralization (i.e., inhibition) of bacterial flagellin, (including flagellin bound to bacteria or “free”, circulating flagellin in the systemic circulation); (ii) cross-reactivity with flagellin from a broad spectrum of bacteria; (iii) inhibition of bacterial invasion into susceptible epithelial cells; (iv) binding to flagellin with an affinity of at least 10¹⁰ M⁻¹; (v) reduction or prevention of flagellin-induced tissue injury; (vi) reduction or prevention of flagellin-stimulated neutrophil infiltration; (vii) reduction or prevention of colonic mucosal congestion, erosion and/or hemorrhagic ulcerations associated with IBD; and (viii) reduction or prevention of cytokine production, including MDA, IL-1β, TNFα, MIP-1, MIP-2, IL-6 and IL-8, and pro-inflammatory free radical synthesizing enzymes, such as the inducible nitric-oxide synthases; (ix) ability to opsonize bacteria; and (x) ability to promote macrophage ingestion of bacteria. U.S. patent application Ser. No. 12/231,777, U.S. patent application Ser. No. 12/231,797, PCT Application No.: PCT/US2008/075387, and PCT Application No. PCT/US2008/075383, teach anti-flagellin antibodies and all are incorporated herein by reference in their entireties.

From a structural standpoint, particular representative antibodies of the invention include a heavy chain variable region comprising an amino acid sequence which is at least 80% (e.g., 85%, 90%, 95%, 96%, 97%, 98% or 99%) identical to the heavy chain variable region amino acid sequence set forth in SEQ ID NO:1. Other particular antibodies of the present invention include a light chain variable region comprising an amino acid sequence which is at least 80% (e.g., 85%, 90%, 95%, 96%, 97%, 98% or 99%) identical to the light chain variable region amino acid sequence set forth in SEQ ID NO:2. The antibodies may include a heavy chain only, a light chain only, or they may also include both of the aforementioned heavy chain and light chain variable regions.

Specifically, in one embodiment, the invention provides an isolated monoclonal antibody that binds to flagellin comprising a heavy chain variable region comprising an amino acid sequence set forth in SEQ ID NO:1 (or an amino acid sequence at least 80% identical thereto). In another embodiment, the invention provides an isolated monoclonal antibody that binds to flagellin comprising a light chain variable region comprising an amino acid sequence set forth in SEQ ID NO:2 (or an amino acid sequence at least 80% identical thereto). In a further embodiment, the invention provides an isolated monoclonal antibody that binds to flagellin comprising a heavy and light chain variable region comprising the amino acid sequences set forth in SEQ ID NOs:1 and 2, respectively (or amino acid sequences at least 80% identical thereto). In yet another embodiment, the invention provides, an isolated monoclonal antibody that binds to flagellin and comprises a heavy chain variable region comprising SEQ ID NO:1, a light chain variable region comprising SEQ ID NO:2, or a combination thereof.

The variable heavy and light chain regions of the antibodies typically include one or more complementarity determining regions (CDRs). These include one or more CDR1, CDR2, and CDR3 regions. Accordingly, other particular antibodies of the present invention include one or more CDR sequences selected from a heavy chain variable region CDR1 comprising SEQ ID NO:5; a heavy chain variable region CDR2 comprising SEQ ID NO:7; a heavy chain variable region CDR3 comprising SEQ ID NO:9; a light chain variable region CDR1 comprising SEQ ID NO:11; a light chain variable region CDR2 comprising SEQ ID NO:13; a light chain variable region CDR3 comprising SEQ ID NO:15; and combinations thereof.

The antibodies may also comprise one or more CDRs which are at least 80% (e.g., 85%, 90%, 95%, 96%, 97%, 98% or 99%) identical to any of the aforementioned CDRs, or combinations of CDRs. For example, in one embodiment, the isolated monoclonal antibody, or antigen binding portion thereof, comprises a heavy chain variable region that comprises CDR1, CDR2, and CDR3 sequences, and a light chain variable region that comprises CDR1, CDR2, and CDR3 sequences, wherein the heavy chain variable region CDR3 sequence comprises SEQ ID NO:9 (or conservative modifications thereof) and the light chain variable region CDR3 sequence comprises SEQ ID NOs:15 (or conservative modifications thereof). In a further embodiment, the antibody comprises the aforementioned CDR3 sequences and, optionally, further comprises a heavy chain variable region CDR2 sequence comprising SEQ ID NO:7 (or conservative modifications thereof), and a light chain variable region CDR2 sequence comprising SEQ ID NO:13 (or conservative modifications thereof). In yet a further embodiment, the antibody comprises the aforementioned CDR3 and CDR2 sequences and, optionally, further comprises a heavy chain variable region CDR1 sequence comprising SEQ ID NO:5 (or conservative modifications thereof), and a light chain variable region CDR1 sequence comprising SEQ ID NO:11 (or conservative modifications thereof).

Also provided by the present invention are antibodies that bind to the same or overlapping epitopes bound by any of the aforementioned antibodies. In a particular embodiment, these antibodies cross-react with a variety of gram-negative bacteria, including Proteus Vulgaris, non-pathogenic E. Coli, Citrobacter freundii, Serratia marcenscens, Enterobacter cloacae, Campylobacter jejuni, Helicobacter pylori, Pseudomonas aeruginosa, Salmonella typhimurium, Salmonella muenchen, Proteus mirabilis and Enteropathogenic E. Coli. In another particular embodiment, the antibodies bind to an epitope on flagellin of Salmonella muenchen which includes all or a portion of (e.g., is located between) amino acids 41-52 (SEQ ID NO:23).

In another aspect, the invention pertains to antibodies that compete for binding to flagellin with the anti-flagellin antibodies described herein. In a particular embodiment, the antibody competes for binding to flagellin with an antibody comprising heavy and/or light chain variable regions comprising the amino acid sequences set forth in SEQ ID NOs:1 and 2, respectively, or amino acid sequences at least 80% identical thereto. Other antibodies of the invention bind to an epitope on flagellin recognized by the antibodies described herein. In another particular embodiment, the antibody binds to an epitope on flagellin recognized by an antibody comprising heavy and/or light chain variable regions comprising the amino acid sequences set forth in SEQ ID NOs:1 and 2, respectively, or amino acid sequences at least 80% identical thereto.

Antibodies of the present invention include all known immunoglobulin forms and other protein scaffolds with antibody-like properties. For example, the antibody can be a murine antibody, a human antibody, a humanized antibody, a chimeric antibody or a protein scaffold with antibody-like properties, such as fibronectin or Ankyrin repeats. The antibody also can have any of the following isotypes: IgG1, IgG2, IgG3, IgG4, IgM, IgA1, IgA2, IgAsec, IgD and IgE. Antibodies of the invention also include antibody fragments, such as an Fab, Fab′2, ScFv, SMIP, affibody, avimer, nanobody or a domain antibody.

Antibodies of the invention can be administered alone or in combination with other therapeutic agents. For example, the antibodies can be administered in combination with (i.e., together with or linked to) cytotoxins, antibacterial agents, including antibiotics and/or other therapeutic antibodies. In one embodiment, the antibody is linked to a second antibody (i.e., thereby forming a bispecific antibody) or other binding agent that binds to a different target (e.g., an Fc receptor on an immune cell) or a different epitope on flagellin.

In yet another aspect, the present invention provides isolated nucleic acids encoding the aforementioned antibodies of the invention. In particular embodiments, the nucleic acid encodes a heavy chain variable region comprising a nucleotide sequence which is at least 80% (e.g., 85%, 90%, 95%, 96%, 97%, 98% or 99%) identical to, or which hybridizes under high stringency conditions to, SEQ ID NO:3; or a light chain variable region comprising a nucleotide sequence which is at least 80% (e.g., 85%, 90%, 95%, 96%, 97%, 98% or 99%) identical to, or which hybridizes under high stringency conditions to, SEQ ID NO:4; or a combination of these heavy and light variable regions.

The present invention also provides hybridomas that express and/or produce the aforementioned antibodies.

Further provided by the invention are kits comprising one or more of the aforementioned antibodies (or immunoconjugates or bispecific antibodies), optionally, with instructions for use in treating or diagnosing bacterial diseases associated with flagellin in humans, other animals and birds.

The present invention also provides methods of producing the particular antibodies of the invention by providing an isolated cell which expresses the antibody of interest and isolating the antibody from the cell, thereby producing the antibody.

As noted above, antibodies of the present invention can be used in a broad variety of diagnostic and therapeutic applications, or used in the manufacture of one or more medicaments for diagnostic or therapeutic applications. These applications include treatment and prevention of both infectious and non-infectious bacterial diseases associated with flagellin. Particular non-infectious diseases include, but are not limited to, inflammatory bowel diseases (IBDs), such as Crohn's Disease and colitis. Other particular diseases include gram negative bacterial infections (e.g., enterobacterial infections) sepsis and septic shock, in particular. Still other particular diseases include Anthrax, Bacterial Meningitis, Botulism, Brucellosis, Cat Scratch Disease, Cholera, Diphtheria, Epidemic Typhus, Impetigo, Legionellosis, Leprosy, Leptospirosis, Listeriosis, Lyme Disease, Melioidosis, MRSA infection, Nocardiosis, Pertussis, Plague, Pneumococcal pneumonia, Psittacosis, Q fever, Rocky Mountain Spotted Fever (RMSF), Salmonellosis, Scarlet Fever, Shigellosis, Syphilis, Tetanus, Trachoma, Tuberculosis, Tularemia, Typhoid Fever, Urinary Tract Infections and Necrotizing enterocolitis.

Other features and advantages of the invention will be apparent from the following detailed description, and from the claims.

### BRIEF DESCRIPTION OF THE DRAWINGS

FIG. 1 shows the results of a Western Blot demonstrating that mAb 743 reacts strongly with Pseudomonas aeruginosa type A flagellin and weakly with Pseudomonas aeruginosa type B flagellin.

FIG. 2 is a graph showing the specific, wide-spread, reactivity of mAb 743 to a variety of gram-negative bacteria in a live bacterial ELISA assay.

FIG. 3 shows the epitope binding region of mAb 743 on Salmonella muenchen flagellin.

FIG. 4 is a graph showing that anti-flagellin mAb 743 inhibits flagellin activity in an NO production assay.

### DETAILED DESCRIPTION OF THE INVENTION

In order that the present invention may be more readily understood, certain terms are first defined. Additional definitions are set forth throughout the detailed description.

### I. Definitions

As used herein, the term “flagellin” carries its art recognized meaning as referring to a monomeric subunit of bacterial flagellum. The term “flagellin” includes the monomeric protein flagellin bound to bacteria, free circulating flagellin, and flagellin subunits of an individual flagellum or flagella. The amino acid sequences of flagellins from different bacterial strains are known in the art and are widely conserved, as discussed by Steiner, T. S. (Infect Immun. 2007 February; 75(2):545-52), the teachings of which are incorporated by reference herein. Preferred antibodies of the invention cross react with flagellins of multiple bacterial species, including, but not limited to, Proteus Vulgaris, non-pathogenic E. Coli, Citrobacter freundii, Serratia marcescens, Pseudomonas aeruginosa, Salmonella typhimurium, Proteus mirabilis, and Enteropathogenic E. Coli. Representative flagellin sequences, include, but are not limited to, the sequences set forth below.

Proteus mirabilis (GI:1169696)

(SEQ ID NO: 19)

MAQVINTNYLSLVTQNNLNKSQGTLGSAIERLSSGLRINSAKDDAAGQAI
ANRFTSNVNGLTQASRNANDGISIAQTTEGALNEINNNLQRIRELTVQAK
NGTNSNSDITSIQNEVKNVLDEINRISEQTQFNGVKVLSGEKSEMVIQVG
TNDNETIKFNLDKVDNDTLGVASDKLFDTKTEKKGVTAAGAGVTDAKKIN
AAATLDMMVSLVKEFNLDGKPVTDKFIVTKGGKDYVATKSDFELDATGTK
LGLKASATTEFKVDAGKDVKTLNVKDDALATLDKAINTIDESRSKLGAIQ
NRFESTINNLNNTVNNLSASRSRILDADYATEVSNMSRGQILQQAGTSVL
AQANQVPQTVLSLLR
(Betas, et al. (1994). Gene 148, 33-41.)
Pseudomonas aeruginosa (GI:3386643)

(SEQ ID NO: 20)

MALTVNTNIASLNTQRNLNNSSASLNTSLQRLSTGSRINSAKDDAAGLQI
ANRLTSQVNGLNVATKNANDGISLAQTAEGALQQSTNILQRMRDLSLQSA
NGSNSDSERTALNGEVKQLQKELDRISNTTTFGGRKLLDGSFGVASFQVG
SAANEIISVGIDEMSAESLNGTYFKADGGGAVTAATASGTVDIAIGITGG
SAVNVKVDMKGNETAEQAAAKIAAAVNDANVGIGAFTDGAQISYVSKASA
DGTTSAVSGVAITDTGSTGAGTAAGTTTFTEANDTVAKIDISTAKGAQSA
VLVIDEAIKQIDAQRADLGAVQNRFDNTINNLKNIGENVSAARGRIEDTD
FAAETANLTKNQVLQQAGTAILAQANQLPQSVLSLLR
(Spangenberg,C. et al., (1996). FEBS Lett. 396,
213-217)
Escherichia coli (GI:1655807)

(SEQ ID NO: 21)

MAQVINTNSLSLITQNNLNKNQSALSSSIERLSSGLRINSAKDDAAGQAI
ANRFTSNIKGLTQAARNANDGISVAQTTEGALSEINNNLQRIRELTVQAT
TGTNSDSDLDSIQDEIKSRLDEIDRVSGQTQFNGVNVLAKDGSMKIQVGA
NDGETITIDLKKIDSDTLGLNGFNVNGKGTITNKAATVSDLTSAGAKLNT
TTGLYDLKTENTLLTTDAAFDKLGNGDKVTVGGVDYTYNAKSGDFTTTKS
TAGTGVDAAAQAADSASKRDALAATLHADVGKSVNGSYTTKDGTVSFETD
SAGNITIGGSQAYVDDAGNLTTNNAGSAAKADMKALLKAASEGSDGASLT
FNGTEYTIAKATPATTTPVAPLIPGGITYQATVSKDVVLSETKAAAATSS
ITFNSGVLSKTIGFTAGESSDAAKSYVDDKGGITNVADYTVSYSVNKDNG
SVTVAGYASATDTNKDYAPAIGTAVNVNSAGKITTETTSAGSATTNPLAA
LDDAISSIDKFRSSLGAIQNRLDSAVTNLNNTTTNLSEAQSRIQDADYAT
EVSNMSKAQIIQQAGNSVLAKANQVPQQVLSLLQG
Serratia marcescens (GI:514988)

(SEQ ID NO: 22)

MAQVINTNSLSLMAQNNLNKSQSSLGTAIERLSSGLRINSAKDDAAGQAI
SDRFTANIKGLTQASRNANDGISLAQTTEGALNEVNDNLQNIRRLTVQAQ
NGSNSTSDLKSIQDEITQRMSEINRISEQTDFNGVKVLSSDQKLTIQVGA
NDGETIDIDLQGLTGFDVTENGTKIGSAIADKAMVKDDTGTDVAFDLGES
FQTGGALEKATLVSGKTKDGKEGYYIQTTDAATGAKTYATAKIDDKGVVT
KGADVTDVKDPLATLDKALAQVDGLRSSLGAVQNRFDSVISNLNSTVNNL
ASQSRIQDADYATEVSNMSRAHILQQAGTSVLAQANQSTQNVLSLLR
(Akatsuka,H. et al., (1995). Gene 163, 157-158)
Salmonella muenchen (GI:1333832)

(SEQ ID NO: 23)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NGTNSQSDLDSIQAEITQRLNEIDRVSGQTQFNGVKVLAQDNTLTIQVGA
NDGETIDIDLKEISSKTLGLDKLNVQDAYTPKETAVTVDKTTYKNGTDTI
TAQSNTDIQTAIGGGATGVTGADIKFKDGQYYLDVKGGASAGVYKATYDE
TTKKVNIDTTDKTPLATAEATAIRGTATITHNQIAEVTKEGVDTTTVAAQ
LAAAGVTGADKDNTSLVKLSFEDKNGKVIDGGYAVKMGDDFYAATYDEKQ
VQLLLNNHYTDGAGVLQTGAVKFGGANGKSEVVTATVGKTYLASDLDKHN
FRTGGELKEVNTDKTENPLQKIDAALAQVDTLRSDLGAVQNRFNSAITNL
GNTVNNLSSARSRIEDSDYATEVSNMSRAQILQQAGTSVLAQANQVPQNV
LSLLR
(Wei, L.N. et al., (1985). J. Mol. Biol. 186,
791-803)
Salmonella typhimurium (GI:153979)

(SEQ ID NO: 24)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVNGQTQFSGVKVLAQDNTLTIQVGA
NDGETIDIDLKQINSQTLGLDTLNVQQKYKVSDTAATVTGYADTTIALDN
STFKASATGLGGTDEKIDGDLKFDDTTGKYYAKVTVTGGTGKDGYYEVSV
DKTNGEVTLAAVTPATVTTATALSGKMYSANPDSDIAKAALTAAGVTGTA
SVVKMSYTDNNGKTIDGGLAVKVGDDYYSATQDKDGSISIDTTKYTADNG
TSKTALNKLGGADGKTEVVTIDGKTYNASKAAGHDFKAEPELAEQAAKTT
ENPLQKIDAALAQVDTLRSDLGAVQNRFNSAITNLGNTVNNLSSARSRIE
DSDYATEVSNMSRAQILQQAGTSVLAQANQVPQNVLSLLR
(Joys,T.M. (1985). J. Biol. Chem. 260, 15758-15761.)

As used herein, the term “bacteria” or “bacterium” refers to unicellular prokaryotic microorganisms, i.e., organisms without a cell nucleus or any other membrane-bound organelles. Bacteria are typically a few micrometres in length and individual bacteria have a wide-range of shapes, ranging from spheres to rods to spirals. Although the vast majority of bacteria are rendered harmless or beneficial by the protective effects of the immune system, a few pathogenic bacteria cause infectious diseases.

As used herein, “gram-negative bacteria” or “gram-negative bacterium” refer to bacteria having characteristic staining properties under the microscope, where they either do not stain or are decolorized by alcohol during Gram's method of staining.

Gram-negative bacteria generally have the following characteristics: (1) their cell wall only contains a few layers of peptidoglycan (which is present in much higher levels in Gram-positive bacteria); (2) the cells are surrounded by an outer membrane containing lipopolysaccharide (which consists of Lipid A, core polysaccharide, and O-polysaccharide) outside the peptidoglycan layer; (3) porins exist in the outer membrane, which act like pores for particular molecules; (4) there is a space between the layers of peptidoglycan and the secondary cell membrane called the periplasmic space; (5) the S-layer is directly attached to the outer membrane, rather than the peptidoglycan; (6) if present, flagella have four supporting rings instead of two; (7) no teichoic acids or lipoteichoic acids are present; (8) lipoproteins are attached to the polysaccharide backbone, whereas in Gram-positive bacteria no lipoproteins are present; and (9) most do not sporulate.

Examples of gram-negative bacteria include, but are not limited to, Escherichia coli, Enterobacteriaceae, Moraxella, Helicobacter, Burkholderia cepacia, Stenotrophomonas, Bdellovibrio, acetic acid bacteria, cyanobacteria, spirochaetes, green sulfur and green non-sulfur bacteria, Neisseria gonorrhoeae, Neisseria meningitides, Moraxella catarrhalis, Hemophilus influenzae, Klebsiella pneumoniae, Legionella pneumophila, Pseudomonas aeruginosa, Proteus mirabilis, Enterobacter cloacae, Serratia marcescens Helicobacter pylori, Salmonella enteritidis, and Salmonella typhi.

As used herein, “a bacterial infectious disease” is a disease or infection caused by bacteria.

As used herein, “a gram negative bacterial infection” is a disease or infection caused by gram negative bacteria.

As used herein, “an enterobacterial infection” is an infection caused by Enterobacteriaceae.

As used herein, “Enterobacteriaceae” and “enterobacteria” refer to a large family of bacteria, including many of the more familiar pathogens, such as Salmonella and Escherichia coli. Genetic studies place them among the Proteobacteria, and they are given their own order (Enterobacteriales). Members of the Enterobacteriaceae are rod-shaped, and are typically 1-5 μm in length. Like other Proteobacteria, they have Gram-negative stains, and they are facultative anaerobes, fermenting sugars to produce lactic acid and various other end products. They also reduce nitrate to nitrite. Unlike most similar bacteria, enterobacteria generally lack cytochrome C oxidase, although there are exceptions (e.g., Plesiomonas). Most have many flagella used to move about, but a few genera are non-motile. They are non-spore forming, and except for Shigella dysenteriae strains they are catalase-positive. Many members of this family are a normal part of the gut flora found in the intestines of humans and other animals, while others are found in water or soil, or are parasites on a variety of different animals and plants.

Examples of Enterobacteriaceae include, but are not limited to, Alishewanella, Alterococcus, Aquamonas, Aranicola, Arsenophonus, Azotivirga, Blochmannia, Brenneria, Buchnera, Budvicia, Buttiauxella, Cedecea, Citrobacter, Dickeya, Edwardsiella, Enterobacter, Erwinia (e.g. Erwinia amylovora), Escherichia (e.g. Escherichia coli), Ewingella, Grimontella, Hafnia, Klebsiella (e.g. Klebsiella pneumoniae), Kluyvera, Leclercia, Leminorella, Moellerella, Morganella, Obesumbacterium, Pantoea, Pectobacterium, Candidatus Phlomobacter, Photorhabdus (e.g. Photorhabdus luminescens), Plesiomonas (e.g. Plesiomonas shigelloides), Pragia Proteus (e.g. Proteus vulgaris), Providencia, Rahnella, Raoultella, Salmonella, Samsonia, Serratia (e.g. Serratia marcescens), Shigella, Sodalis, Tatumella, Trabulsiella, Wigglesworthia, Xenorhabdus, Yersinia (e.g. Yersinia pestis), and Yokenella.

Examples of enterobacterial infections include, but are not limited to, Anthrax (by the bacterium Bacillus anthracis), Bacterial Meningitis (caused by a variety of bacteria, including, but not limited to, Neisseria meningitides, Streptococcus pneumoniae, Listeria monocytogenes, Pseudomonas aeruginosa, Staphylococcus aureus, Streptococcus agalactiae and Haemophilus influenzae), Botulism (caused by bacterium Clostridium botulinum), Brucellosis (caused by bacteria of the genus Brucella), Campylobacteriosis (caused by bacteria of the genus Campylobacter), Cat Scratch Disease (caused by Bartonella henselae and Bartonella clarridgeiae), Cholera (caused by the bacterium Vibrio cholerae), Diphtheria (caused by Corynebacterium diphtheriae), Epidemic Typhus (causative organism is Rickettsia prowazekii), Impetigo (caused by several bacteria, including, Staphylococcus aureus and Streptococcus pyogenes), Legionellosis (caused by bacteria belonging to the genus Legionella), Leprosy (Hansen's Disease) (caused by the bacterium Mycobacterium leprae), Leptospirosis (caused by spirochaetes of the genus Leptospira), Listeriosis (caused by the bacterium Listeria monocytogenes), Lyme Disease (caused by spirochete bacteria from the genus Borrelia), Melioidosis (caused by the bacterium Burkholderia pseudomallei), MRSA infection (caused by Staphylococcus aureus), Nocardiosis (bacterium of the genus Nocardia, most commonly Nocardia asteroides or Nocardia brasiliensis), Pertussis (Whooping Cough) (caused by the bacterium Bordetella pertussis), Plague (caused by the enterobacteria Yersinia pestis), Pneumococcal pneumonia (caused by a variety of bacteria, including, but not limited to, Streptococcus pneumoniae, Staphylococcus aureus, Haemophilus influenzae, Klebsiella pneumoniae, Escherichia coli, Pseudomonas aeruginosa, Moraxella catarrhalis, Chlamydophila pneumoniae, Mycoplasma pneumoniae, and Legionella pneumophila), Psittacosis (caused by a bacterium called Chlamydophila psittaci), Q fever (caused by infection with Coxiella burnetii), Rocky Mountain Spotted Fever (RMSF) (by Rickettsia rickettsii), Salmonellosis (caused by bacteria of the genus Salmonella), Scarlet Fever, Shigellosis (caused by bacteria of the genus Shigella), Syphilis (caused by Treponema pallidum), Tetanus (Clostridium tetani), Trachoma, Tuberculosis (caused by mycobacteria, mainly Mycobacterium tuberculosis), Tularemia (by the bacterium Francisella tularensis), Typhoid Fever (caused by the bacterium Salmonella typhi), and Urinary Tract Infections (caused by bacteria such as Escherichia coli, Staphylococcus saprophyticus, Proteus mirabilis, Klebsiella pneumoniae, Enterobacter spp., Pseudomonas and Enterococcus).

As used herein, “gram-positive bacteria” or “gram-positive bacterium” refer to bacteria that retain the stain or that are resistant to decolourisation by alcohol during Gram's method of staining. Gram-positive bacteria generally have the following characteristics: (1) a very thick cell wall (peptidoglycan); (2) if a flagellum is present, it contains two rings for support as opposed to four in Gram-negative bacteria because Gram-positive bacteria have only one membrane layer; and (3) teichoic acids and lipoteichoic acids are present, which serve to act as chelating agents, and also for certain types of adherence. Examples of gram-positive bacteria genera include, but are not limited to, Bacillus, Listeria, Staphylococcus, Streptococcus, Enterococcus, and Clostridium.

As used herein, “Inflammatory Bowel Disease (IBD)” refers to a group of chronic intestinal diseases characterized by inflammation of the bowel, i.e., the large or small intestine. The most common types of IBD are Ulcerative Colitis and Crohn's Disease. The symptoms of IBD include abdominal pain, diarrhea, bloody diarrhea, severe urgency to have a bowel movement, fever, loss of appetite, weight loss, anemia. IBD can also cause intestinal complications including profuse bleeding from the ulcers, perforation of the bowel, strictures and obstructions, fistulae, perianal disease, toxic megacolon and cancer. The disease can be limited to the intestine or affect the skin, joints, spine, liver, eyes, and other organs.

As used herein, “Crohn's Disease” is a form of IBD that causes severe irritation in the gastrointestinal tract. It usually affects the lower small intestine (i.e., the ileum) or the colon, but can affect other parts of the digestive system including the small intestine, mouth, esophagus, and stomach. The inflammation in Crohn's Disease involves the entire thickness of the bowel wall. There are five different types of Crohn's disease: (1) Ileocolitis (the most common form, which affects the ileum and the colon); (2) Ileitis (which affects the ileum); (3) Gastroduodenal Crohn's Disease (which causes inflammation in the stomach and the duodenum); (4) Jejunoileitis (which causes spotty patches of inflammation in the top half of the small intestine (i.e., the jejunum); and (5) Crohn's (Granulomatous) Colitis (which affects only affects the large intestine).

As used herein, “Ulcerative Colitis” is a form of IBD that affects the colon (the large intestine) alone and inflammation is confined to the mucosa (the inner lining) of the intestine. It can be difficult to diagnose because its symptoms are similar to other intestinal disorders and Crohn's Disease.

The term “Toll-like receptor (TLR)” as used herein, refers to an important family of innate immune receptors that recognize pathogen-associated molecular patterns, i.e., evolutionarily conserved structures that are required for microbial fitness and are not present in the host.

The term “Toll-like receptor 5 (TLR5)” as used herein, refers to the Toll-like receptor which recognizes and binds bacterial flagellin from both gram-positive and gram-negative and activates host inflammatory responses. TLR5 is specifically expressed in monocytes, immature dendritic cells and epithelial cells.

The term “neutralizes” and “inhibits” are used interchangeably herein, and refer to any statistically significant decrease in the biological activity (e.g., motility) of flagellin, including full blocking of the activity. For example, “neutralizes” or “inhibits” can refer to a decrease of about 10%, 15%, 20%, 25%, 30%, 35%, 40%, 45%, 50%, 55% 60%, 65%, 70%, 75%, 80%, 85%, 90%, 95%, 98%, 99%, or 100% in flagellin activity.

In particular embodiments of the invention, neutralization or inhibition of flagellin activity results in one or more of the following effects: it prevents bacterial invasion into susceptible epithelial cells, reduces the symptoms of an enterobacterial infection or IBD in a subject, reduces the extent and severity of flagellin-induced tissue injury, reduces flagellin-stimulated neutrophil infiltration, decreases colonic mucosal congestion, erosion and hemorrhagic ulcerations associated with IBD, inhibits or decrease the production of mediators (e.g., MDA, IL-1β, TNFα, MIP-1, MIP-2 and IL-8); and/or counteracts a reduction in body weight associated with IBD.

The term “antibody” or “immunoglobulin,” as used interchangeably herein, includes whole antibodies and any antigen binding fragment (i.e., “antigen-binding portion”) or single chains thereof. An “antibody” comprises at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or simply “antibody portion”), as used herein, refers to one or more fragments of an antibody that retain the ability to specifically bind to an antigen (e.g., flagellin). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, CL and CH1 domains; (ii) a F(ab′)₂ fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb including VH and VL domains; (vi) a dAb fragment (Ward et al. (1989) Nature 341, 544-546), which consists of a V_{H} domain; (vii) a dAb which consists of a VH or a VL domain; and (viii) an isolated complementarity determining region (CDR) or (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242, 423-426; and Huston et al. (1988) Proc. Natl. Acad. Sci. USA 85, 5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. Monoclonal antibodies can be prepared using any art recognized technique and those described herein such as, for example, a hybridoma method, as described by Kohler et al. (1975) Nature, 256:495, a transgenic animal, as described by, for example, (see e.g., Lonberg, et al. (1994) Nature 368(6474): 856-859), recombinant DNA methods (see, e.g., U.S. Pat. No. 4,816,567), or using phage antibody libraries using the techniques described in, for example, Clackson et al., Nature, 352:624-628 (1991) and Marks et al., J. Mol. Biol., 222:581-597 (1991). Monoclonal antibodies include chimeric antibodies, human antibodies and humanized antibodies and may occur naturally or be recombinantly produced.

The term “recombinant antibody,” refers to antibodies that are prepared, expressed, created or isolated by recombinant means, such as (a) antibodies isolated from an animal (e.g., a mouse) that is transgenic or transchromosomal for immunoglobulin genes (e.g., human immunoglobulin genes) or a hybridoma prepared therefrom, (b) antibodies isolated from a host cell transformed to express the antibody, e.g., from a transfectoma, (c) antibodies isolated from a recombinant, combinatorial antibody library (e.g., containing human antibody sequences) using phage display, and (d) antibodies prepared, expressed, created or isolated by any other means that involve splicing of immunoglobulin gene sequences (e.g., human immunoglobulin genes) to other DNA sequences. Such recombinant antibodies may have variable and constant regions derived from human germline immunoglobulin sequences. In certain embodiments, however, such recombinant human antibodies can be subjected to in vitro mutagenesis and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The term “chimeric immunoglobulin” or antibody refers to an immunoglobulin or antibody whose variable regions derive from a first species and whose constant regions derive from a second species. Chimeric immunoglobulins or antibodies can be constructed, for example by genetic engineering, from immunoglobulin gene segments belonging to different species.

The term “human antibody,” as used herein, is intended to include antibodies having variable regions in which both the framework and CDR regions are derived from human germline immunoglobulin sequences as described, for example, by Kabat et al. (See Kabat, et al. (1991) Sequences of proteins of Immunological Interest, Fifth Edition, U.S. Department of Health and Human Services, NIH Publication No. 91-3242). Furthermore, if the antibody contains a constant region, the constant region also is derived from human germline immunoglobulin sequences. The human antibodies may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The human antibody can have at least one or more amino acids replaced with an amino acid residue, e.g., an activity enhancing amino acid residue which is not encoded by the human germline immunoglobulin sequence. Typically, the human antibody can have up to twenty positions replaced with amino acid residues which are not part of the human germline immunoglobulin sequence. In a particular embodiment, these replacements are within the CDR regions as described in detail below.

The term “humanized immunoglobulin” or “humanized antibody” refers to an immunoglobulin or antibody that includes at least one humanized immunoglobulin or antibody chain (i.e., at least one humanized light or heavy chain). The term “humanized immunoglobulin chain” or “humanized antibody chain” (i.e., a “humanized immunoglobulin light chain” or “humanized immunoglobulin heavy chain”) refers to an immunoglobulin or antibody chain (i.e., a light or heavy chain, respectively) having a variable region that includes a variable framework region substantially from a human immunoglobulin or antibody and complementarity determining regions (CDRs) (e.g., at least one CDR, preferably two CDRs, more preferably three CDRs) substantially from a non-human immunoglobulin or antibody, and further includes constant regions (e.g., at least one constant region or portion thereof, in the case of a light chain, and preferably three constant regions in the case of a heavy chain). The term “humanized variable region” (e.g., “humanized light chain variable region” or “humanized heavy chain variable region”) refers to a variable region that includes a variable framework region substantially from a human immunoglobulin or antibody and complementarity determining regions (CDRs) substantially from a non-human immunoglobulin or antibody.

A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, (1990) Clin. Exp. Immunol. 79, 315-321; Kostelny et al. (1992) J. Immunol. 148, 1547-1553.

As used herein, a “heterologous antibody” is defined in relation to the transgenic non-human organism or plant producing such an antibody.

An “isolated antibody,” as used herein, is intended to refer to an antibody which is substantially free of other antibodies having different antigenic specificities (e.g., an isolated antibody that specifically binds to flagellin is substantially free of antibodies that specifically bind antigens other than flagellin). In addition, an isolated antibody is typically substantially free of other cellular material and/or chemicals. In one embodiment of the invention, a combination of “isolated” monoclonal antibodies having different flagellin binding specificities are combined in a well defined composition.

As used herein, “isotype” refers to the antibody class (e.g., IgM or IgG1) that is encoded by heavy chain constant region genes. In one embodiment, an antibody or antigen binding portion thereof is of an isotype selected from an IgG1, an IgG2, an IgG3, an IgG4, an IgM, an IgA1, an IgA2, an IgAsec, an IgD, or an IgE antibody isotype. In some embodiments, a monoclonal antibody of the invention is of the IgG1 isotype. In other embodiments, a monoclonal antibody of the invention is of the IgG2 isotype.

As used herein, “isotype switching” refers to the phenomenon by which the class, or isotype, of an antibody changes from one Ig class to one of the other Ig classes.

As used herein, “nonswitched isotype” refers to the isotypic class of heavy chain that is produced when no isotype switching has taken place; the CH gene encoding the nonswitched isotype is typically the first CH gene immediately downstream from the functionally rearranged VDJ gene. Isotype switching has been classified as classical or non-classical isotype switching. Classical isotype switching occurs by recombination events which involve at least one switch sequence regions in a gene encoding an antibody. Non-classical isotype switching may occur by, for example, homologous recombination between human σ_{μ} and human Σ_{μ} (δ-associated deletion). Alternative non-classical switching mechanisms, such as intertransgene and/or interchromosomal recombination, among others, may occur and effectuate isotype switching.

As used herein, the term “switch sequence” refers to those DNA sequences responsible for switch recombination. A “switch donor” sequence, typically a μ switch region, will be 5′ (i.e., upstream) of the construct region to be deleted during the switch recombination. The “switch acceptor” region will be between the construct region to be deleted and the replacement constant region (e.g., γ, ε, etc.). As there is no specific site where recombination always occurs, the final gene sequence will typically not be predictable from the construct.

An “antigen” is an entity (e.g., a proteinaceous entity or peptide) to which an antibody or antigen-binding portion thereof binds. In various embodiments of the present invention, an antigen is flagellin.

The term “epitope” or “antigenic determinant” refers to a site on an antigen to which an immunoglobulin or antibody specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody (i.e., epitope mapping) are well known in the art and include, for example, immunoblotting and immunoprecipitation assays, wherein overlapping or contiguous peptides from flagellin are tested for reactivity with the given anti-flagellin antibody. Methods of determining spatial conformation of epitopes are also well known in the art and include, for example, x-ray crystallography and 2-dimensional nuclear magnetic resonance. See, e.g., Epitope Mapping Protocols in Methods in Molecular Biology, Vol. 66, G. E. Morris, Ed. (1996).

Accordingly, also encompassed by the present invention are antibodies that bind to an epitope on flagellin which comprises all or a portion of an epitope recognized by the particular antibodies described herein (e.g., the same or an overlapping region or a region between or spanning the region). In a particular embodiment, the antibodies bind to an epitope which comprises all or a portion of amino acids 1-60 of flagellin from Salmonella (Genbank Accession No. GI:1333832) (SEQ ID NO:23) or Pseudomonas (Genbank Accession No. GI:3386643) (SEQ ID NO:20), or any other homologous flagellin sequences from various bacterial species, such as an epitope that is between or includes all or a portion of amino acids 1-30 or 1-40 or 1-50 or 1-55 or 20-40 or 30-40 or 30-50 or 35-45 or 37-43 or 31-47 or 41-52 or 45-55 or 40-55 or 40-60 of flagellin from Salmonella (Genbank Accession No. GI:1333832) (SEQ ID NO:23) or Pseudomonas (Genbank Accession No. GI:3386643) (SEQ ID NO:20). In a particular embodiment, the antibodies bind to an epitope which comprises all or a portion of (i.e., is located between, spans, or overlaps) with amino acids 41-53 of Salmonella muenchen flagellin (SEQ ID NO:23).

In another embodiment, the invention provides antibodies that compete for binding to flagellin (e.g., Salmonella muenchen flagellin (SEQ ID NO:23)) with the antibodies described herein. Competing antibodies and antibodies that recognize the same or an overlapping epitope can be identified using routine techniques such as an immunoassay, for example, by showing the ability of one antibody to block the binding of another antibody to a target antigen, i.e., a competitive binding assay. Competitive binding is determined in an assay in which the immunoglobulin under test inhibits specific binding of a reference antibody to an antigen, such as flagellin. Numerous types of competitive binding assays are known, for example: solid phase direct or indirect radioimmunoassay (RIA), solid phase direct or indirect enzyme immunoassay (EIA), sandwich competition assay (see Stahli et al., (1983) Methods in Enzymology 9:242); solid phase direct biotin-avidin EIA (see Kirkland et al., (1986) J. Immunol. 137:3614); solid phase direct labeled assay, solid phase direct labeled sandwich assay (see Harlow and Lane, (1988) Antibodies: A Laboratory Manual, Cold Spring Harbor Press); solid phase direct label RIA using I-125 label (see Morel et al., (1988) Mol. Immunol. 25(1):7); solid phase direct biotin-avidin EIA (Cheung et al., (1990) Virology 176:546); and direct labeled RIA. (Moldenhauer et al., (1990) Scand. J. Immunol. 32:77). Typically, such an assay involves the use of purified antigen (e.g., flagellin) bound to a solid surface or cells bearing either of these, an unlabeled test immunoglobulin and a labeled reference immunoglobulin. Competitive inhibition is measured by determining the amount of label bound to the solid surface or cells in the presence of the test immunoglobulin. Usually the test immunoglobulin is present in excess. Usually, when a competing antibody is present in excess, it will inhibit specific binding of a reference antibody to a common antigen by at least 50-55%, 55-60%, 60-65%, 65-70% 70-75% or more.

As used herein, the terms “specific binding,” “specifically binds,” “selective binding,” and “selectively binds,” mean that an antibody or antigen-binding portion thereof, exhibits appreciable affinity for a particular antigen or epitope and, generally, does not exhibit significant cross-reactivity with other antigens and epitopes. “Appreciable” or preferred binding includes binding with an affinity of at least 10⁶, 10⁷, 10⁸, 10⁹ M⁻¹, or 10¹⁰ M⁻¹. Affinities greater than 10⁷M⁻¹, preferably greater than 10⁸ M⁻¹ are more preferred. Values intermediate of those set forth herein are also intended to be within the scope of the present invention and a preferred binding affinity can be indicated as a range of affinities, for example, 10⁶ to 10¹⁰ M⁻¹, preferably 10⁷ to 10¹⁰ M⁻¹, more preferably 10⁸ to 10¹⁰ M⁻¹. An antibody that “does not exhibit significant cross-reactivity” is one that will not appreciably bind to an undesirable entity (e.g., an undesirable proteinaceous entity). Specific or selective binding can be determined according to any art-recognized means for determining such binding, including, for example, according to Scatchard analysis and/or competitive binding assays.

The term “K_{D},” as used herein, is intended to refer to the dissociation equilibrium constant of a particular antibody-antigen interaction or the affinity of an antibody for an antigen. In one embodiment, the antibody or antigen binding portion thereof according to the present invention binds an antigen (e.g., flagellin) with an affinity (K_{D}) of 50 nM or better (i.e., or less) (e.g., 40 nM or 30 nM or 20 nM or 10 nM or less), as measured using a surface plasmon resonance assay or a cell binding assay. In a particular embodiment, an antibody or antigen binding portion thereof according to the present invention binds flagellin with an affinity (K_{D}) of 8 nM or better (e.g., 7 nM, 6 nM, 5 nM, 4 nM, 2 nM, 1.5 nM, 1.4 nM, 1.3 nM, 1 nM or less), as measured by a surface plasmon resonance assay or a cell binding assay. In other embodiments, an antibody or antigen binding portion thereof binds an antigen (e.g., flagellin) with an affinity (K_{D}) of approximately less than 10⁻⁷ M, such as approximately less than 10⁻⁸ M, 10⁻⁹ M or 10⁻¹⁰ M or even lower when determined by surface plasmon resonance (SPR) technology in a BIACORE 3000 instrument using recombinant flagellin as the analyte and the antibody as the ligand, and binds to the predetermined antigen with an affinity that is at least two-fold greater than its affinity for binding to a non-specific antigen (e.g., BSA, casein) other than the predetermined antigen or a closely-related antigen.

The term “K_{off},” as used herein, is intended to refer to the off rate constant for the dissociation of an antibody from the antibody/antigen complex.

The term “EC50,” as used herein, refers to the concentration of an antibody or an antigen-binding portion thereof, which induces a response, either in an in vitro or an in vivo assay, which is 50% of the maximal response, i.e., halfway between the maximal response and the baseline.

As used herein, “glycosylation pattern” is defined as the pattern of carbohydrate units that are covalently attached to a protein, more specifically to an immunoglobulin protein.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

The term “rearranged” as used herein refers to a configuration of a heavy chain or light chain immunoglobulin locus wherein a V segment is positioned immediately adjacent to a D-J or J segment in a conformation encoding essentially a complete V_{H} or V_{L} domain, respectively. A rearranged immunoglobulin gene locus can be identified by comparison to germline DNA; a rearranged locus will have at least one recombined heptamer/nonamer homology element.

The term “unrearranged” or “germline configuration” as used herein in reference to a V segment refers to the configuration wherein the V segment is not recombined so as to be immediately adjacent to a D or J segment.

The term “nucleic acid molecule,” as used herein, is intended to include DNA molecules and RNA molecules. A nucleic acid molecule may be single-stranded or double-stranded, but preferably is double-stranded DNA.

The term “isolated nucleic acid molecule,” as used herein in reference to nucleic acids encoding antibodies (e.g., V_{H}, V_{L}, CDR3) that bind to flagellin, is intended to refer to a nucleic acid molecule in which the nucleotide sequences encoding the antibody are free of other nucleotide sequences encoding antibodies that bind antigens other than flagellin, which other sequences may naturally flank the nucleic acid in human genomic DNA.

Alternatively, antibodies can comprise an amino acid sequence which is encoded by a nucleotide sequence which hybridizes, e.g., hybridizes under stringent conditions to a nucleotide sequence disclosed herein. As used herein, the term “hybridizes under stringent conditions” is intended to describe conditions for hybridization and washing under which nucleotide sequences at least 60% homologous to each other typically remain hybridized to each other. Preferably, the conditions are such that sequences at least about 65%, more preferably at least about 70%, and even more preferably at least about 75% or more homologous to each other typically remain hybridized to each other. Such stringent conditions are known to those of ordinary skill in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2×SSC, 0.1% SDS at 50-65° C.

The term “modifying,” or “modification,” as used herein, is intended to refer to changing one or more amino acids in the antibodies. The change can be produced by adding, substituting or deleting an amino acid at one or more positions. The change can be produced using known techniques, such as PCR mutagenesis. For example, in some embodiments, an antibody identified using the methods of the invention can be modified, to thereby modify the binding affinity of the antibody to flagellin.

The present invention also encompasses “conservative amino acid substitutions” in the sequences of the antibodies of the invention, i.e., nucleotide and amino acid sequence modifications which do not abrogate the binding of the antibody encoded by the nucleotide sequence or containing the amino acid sequence, to the antigen, i.e., flagellin. Conservative amino acid substitutions include the substitution of an amino acid in one class by an amino acid of the same class, where a class is defined by common physicochemical amino acid side chain properties and high substitution frequencies in homologous proteins found in nature, as determined, for example, by a standard Dayhoff frequency exchange matrix or BLOSUM matrix. Six general classes of amino acid side chains have been categorized and include: Class I (Cys); Class II (Ser, Thr, Pro, Ala, Gly); Class III (Asn, Asp, Gln, Glu); Class IV (His, Arg, Lys); Class V (Ile, Leu, Val, Met); and Class VI (Phe, Tyr, Trp). For example, substitution of an Asp for another class III residue such as Asn, Gln, or Glu, is a conservative substitution. Thus, a predicted nonessential amino acid residue in an anti-flagellin antibody of the present invention is preferably replaced with another amino acid residue from the same class. Methods of identifying nucleotide and amino acid conservative substitutions which do not eliminate antigen binding are well-known in the art (see, e.g., Brummell et al., Biochem. 32:1180-1187 (1993); Kobayashi et al. Protein Eng. 12(10):879-884 (1999); and Burks et al. Proc. Natl. Acad. Sci. USA 94:412-417 (1997)).

The term “non-conservative amino acid substitution” refers to the substitution of an amino acid in one class with an amino acid from another class; for example, substitution of an Ala, a class II residue, with a class III residue such as Asp, Asn, Glu, or Gln.

Alternatively, in another embodiment, mutations (conservative or non-conservative) can be introduced randomly along all or part of an anti-flagellin antibody coding sequence, such as by saturation mutagenesis, and the resulting modified anti-flagellin antibodies can be screened for binding activity.

A “consensus sequence” is a sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” of an immunoglobulin refers to a framework region in the consensus immunoglobulin sequence.

Similarly, the consensus sequence for the CDRs can be derived by optimal alignment of the CDR amino acid sequences of flagellin antibodies of the present invention.

For nucleic acids, the term “substantial homology” indicates that two nucleic acids, or designated sequences thereof, when optimally aligned and compared, are identical, with appropriate nucleotide insertions or deletions, in at least about 80% of the nucleotides, usually at least about 90% to 95%, and more preferably at least about 98% to 99.5% of the nucleotides. Alternatively, substantial homology exists when the segments will hybridize under selective hybridization conditions, to the complement of the strand.

The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software, using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences of the present invention can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the nucleic acid molecules of the invention. BLAST protein searches can be performed with the XBLAST program, score=50, wordlength=3 to obtain amino acid sequences homologous to the protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al., ed. Current Protocols in Molecular Biology, Greene Publishing and Wiley Interscience, New York (1987).

The nucleic acid compositions of the present invention, while often in a native sequence (except for modified restriction sites and the like), from either cDNA, genomic or mixtures thereof may be mutated, in accordance with standard techniques to provide gene sequences. For coding sequences, these mutations, may affect amino acid sequence as desired. In particular, DNA sequences substantially homologous to or derived from native V, D, J, constant, switches and other such sequences described herein are contemplated (where “derived” indicates that a sequence is identical or modified from another sequence).

The term “operably linked” refers to a nucleic acid sequence placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice. A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. The terms, “plasmid” and “vector” may be used interchangeably. However, the invention is intended to include such other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The terms “treat,” “treating,” and “treatment,” as used herein, refer to therapeutic or preventative measures described herein. The methods of “treatment” employ administration to a subject, an antibody of the present invention, for example, a subject having an infection or disease associated with flagellin or predisposed to having such an infection or disease, in order to prevent, cure, delay, reduce the severity of, or ameliorate one or more symptoms of the infection or disease in order to prolong the survival of a subject beyond that expected in the absence of such treatment.

The terms “effective amount” and “therapeutically effective amount” as used herein, refers to that amount of an antibody thereof that binds flagellin, which is sufficient to effect treatment, prognosis or diagnosis of an infection or disease associated with flagellin, as described herein, when administered to a subject. A therapeutically effective amount will vary depending upon the subject and the infection or disease condition being treated, the weight and age of the subject, the severity of the infection or disease condition, the manner of administration and the like, which can readily be determined by one of ordinary skill in the art. The dosages for administration can range from, for example, about 1 ng to about 10,000 mg, about 5 ng to about 9,500 mg, about 10 ng to about 9,000 mg, about 20 ng to about 8,500 mg, about 30 ng to about 7,500 mg, about 40 ng to about 7,000 mg, about 50 ng to about 6,500 mg, about 100 ng to about 6,000 mg, about 200 ng to about 5,500 mg, about 300 ng to about 5,000 mg, about 400 ng to about 4,500 mg, about 500 ng to about 4,000 mg, about 1 μg to about 3,500 mg, about 5 μg to about 3,000 mg, about 10 μg to about 2,600 mg, about 20 μg to about 2,575 mg, about 30 μg to about 2,550 mg, about 40 μg to about 2,500 mg, about 50 μg to about 2,475 mg, about 100 μg to about 2,450 mg, about 200 μg to about 2,425 mg, about 300 μg to about 2,000, about 400 μg to about 1,175 mg, about 500 μg to about 1,150 mg, about 0.5 mg to about 1,125 mg, about 1 mg to about 1,100 mg, about 1.25 mg to about 1,075 mg, about 1.5 mg to about 1,050 mg, about 2.0 mg to about 1,025 mg, about 2.5 mg to about 1,000 mg, about 3.0 mg to about 975 mg, about 3.5 mg to about 950 mg, about 4.0 mg to about 925 mg, about 4.5 mg to about 900 mg, about 5 mg to about 875 mg, about 10 mg to about 850 mg, about 20 mg to about 825 mg, about 30 mg to about 800 mg, about 40 mg to about 775 mg, about 50 mg to about 750 mg, about 100 mg to about 725 mg, about 200 mg to about 700 mg, about 300 mg to about 675 mg, about 400 mg to about 650 mg, about 500 mg, or about 525 mg to about 625 mg, of an antibody of the present invention. Dosage regimens may be adjusted to provide the optimum therapeutic response. An effective amount is also one in which any toxic or detrimental effects (i.e., side effects) of an antibody are minimized and/or outweighed by the beneficial effects.

The term “patient” includes human and other mammalian subjects that receive either prophylactic or therapeutic treatment.

As used herein, the term “subject” includes any human or non-human animal. For example, the methods and compositions of the present invention can be used to treat a subject having a bacterial disease. In a particular embodiment, the subject is a human. The term “non-human animal” includes all vertebrates, e.g., mammals and non-mammals, such as non-human primates, sheep, rabbits, dogs, cows, chickens, amphibians, reptiles, etc.

The term “sample” refers to tissue, body fluid, or a cell from a patient or a subject. Normally, the tissue or cell will be removed from the patient, but in vivo diagnosis is also contemplated. Other patient samples, include blood, urine, tear drops, serum, cerebrospinal fluid, feces, sputum, cell extracts, lymph, gynecological fluid, ocular fluid, and fluid collected by peritoneal rinsing, etc.

The term “therapeutic agent” refers to any agent which acts in conjunction with or synergistically with the antibody to treat or prevent an infection-associated infection or disease. Therapeutic agents include, but are not limited to, chemotherapeutic agents, cytotoxic agents, anti-inflammatory agents, e.g., a steroidal or nonsteroidal inflammatory agent, or a cytotoxin antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The term “cytotoxin” or “cytotoxic agent” includes any agent that is detrimental to (e.g., kills) cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

Various aspects of the invention are described in further detail in the following subsections.

### II. Methods for Producing Anti-Flagellin Antibodies

(i) Monoclonal Antibodies

Monoclonal antibodies of the invention can be produced using a variety of known techniques, such as those described in the examples, as well as the standard somatic cell hybridization technique described by Kohler and Milstein (1975) Nature 256: 495, viral or oncogenic transformation of B lymphocytes or phage display technique using libraries of human antibody genes. In particular embodiments, the antibodies are fully human monoclonal antibodies.

Accordingly, in one embodiment, a hybridoma method is used for producing an antibody that binds flagellin. In this method, a mouse or other appropriate host animal can be immunized with flagellin protein (or a fragment of flagellin) in order to elicit lymphocytes that produce or are capable of producing antibodies that will specifically bind to this antigen. Suitable flagellin protein can be obtained using a variety of methods, purified from a source, produced recombinantly or chemically synthesized. In a particular embodiment of the present invention, antibodies are raised against flagellin from Salmonella (Genbank Accession No. GI:1333832) (SEQ ID NO:23) or Pseudomonas (Genbank Accession No. GI:3386643) (SEQ ID NO:20).

Alternatively, lymphocytes may be immunized in vitro. Lymphocytes can then be fused with myeloma cells using a suitable fusing agent, such as polyethylene glycol, to form a hybridoma cell (Goding, Monoclonal Antibodies: Principles and Practice, pp. 59-103 (Academic Press, 1986)). Culture medium in which hybridoma cells are growing is assayed for production of monoclonal antibodies directed against the antigen. After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal. The monoclonal antibodies secreted by the subclones can be separated from the culture medium, ascites fluid, or serum by conventional immunoglobulin purification procedures such as, for example, protein A-Sepharose, hydroxylapatite chromatography, gel electrophoresis, dialysis, or affinity chromatography.

In another embodiment, antibodies (and binding fragments thereof) that bind flagellin can be isolated from antibody phage libraries generated using the techniques described in, for example, McCafferty et al., Nature, 348:552-554 (1990). Clackson et al., Nature, 352:624-628 (1991), Marks et al., J. Mol. Biol., 222:581-597 (1991) and Hoet et al (2005) Nature Biotechnology 23, 344-348; U.S. Pat. Nos. 5,223,409; 5,403,484; and 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al. Additionally, production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)), may also be used.

In a particular embodiment, the antibodies of the invention are fully human antibodies. Such antibodies can be produced using a variety of known methods, for example, the phage display technique described by Hoet et al., supra. This technique involves the generation of a human Fab library having a unique combination of immunoglobulin sequences isolated from human donors and having synthetic diversity in the heavy-chain CDRs is generated. The library is then screened for Fabs that bind to flagellin.

Additionally, fully human antibodies directed against flagellin can be generated using transgenic or transchromosomic mice carrying parts of the human immune system rather than the mouse system (see e.g., Lonberg, et al. (1994) Nature 368(6474): 856-859; Lonberg, N. et al. (1994), supra; reviewed in Lonberg, N. (1994) Handbook of Experimental Pharmacology 113:49-101; Lonberg, N. and Huszar, D. (1995) Intern. Rev. Immunol. 13: 65-93, and Harding, F. and Lonberg, N. (1995) Ann. N.Y. Acad. Sci. 764:536-546. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Publication Nos. WO 92/03918, WO 93/12227, WO 94/25585, WO 97/13852, WO 98/24884 and WO 99/45962, all to Lonberg and Kay; and PCT Publication No. WO 01/14424 to Korman et al.).

Other techniques for generating fully human antibodies of the invention include the use of a mouse that carries human immunoglobulin sequences on transgenes and transchomosomes, such as a mouse that carries a human heavy chain transgene and a human light chain transchromosome (see e.g., PCT Publication WO 02/43478 to Ishida et al.).

Still further, alternative transgenic animal systems expressing human immunoglobulin genes are available in the art and can be used to raise anti-flagellin antibodies of the invention. For example, an alternative transgenic system referred to as the Xenomouse (Abgenix, Inc.) can be used; such mice are described in, for example, U.S. Pat. Nos. 5,939,598; 6,075,181; 6,114,598; 6,150,584 and 6,162,963 to Kucherlapati et al.

Moreover, alternative transchromosomic animal systems expressing human immunoglobulin genes are available in the art and can be used to raise anti-flagellin antibodies of the invention. For example, mice carrying both a human heavy chain transchromosome and a human light chain transchromosome can be used; as described in Tomizuka et al. (2000) Proc. Natl. Acad. Sci. USA 97:722-727. Furthermore, cows carrying human heavy and light chain transchromosomes have been described in the art (Kuroiwa et al. (2002) Nature Biotechnology 20:889-894) and can be used to raise antibodies of the present invention.

In yet another embodiment, antibodies of the present invention can be prepared using a transgenic plant and/or cultured plant cells (such as, for example, tobacco, maize and duckweed) that produce such antibodies. For example, transgenic tobacco leaves expressing antibodies or antigen binding portions thereof can be used to produce such antibodies by, for example, using an inducible promoter (see, e.g., Cramer et al., Curr. Top. Microbol. Immunol. 240:95 118 (1999)). Also, transgenic maize can be used to express such antibodies and antigen binding portions thereof (see, e.g., Hood et al., Adv. Exp. Med. Biol. 464:127 147 (1999)). Antibodies can also be produced in large amounts from transgenic plant seeds including antibody portions, such as single chain antibodies (scFv's), for example, using tobacco seeds and potato tubers (see, e.g., Conrad et al., Plant Mol. Biol. 38:101 109 (1998)). Methods of producing antibodies or antigen binding portions in plants can also be found in, e.g., Fischer et al., Biotechnol. Appl. Biochem. 30:99 108 (1999), Ma et al., Trends Biotechnol. 13:522 7 (1995); Ma et al., Plant Physiol. 109:341 6 (1995); Whitelam et al., Biochem. Soc. Trans. 22:940 944 (1994) and U.S. Pat. Nos. 6,040,498 and 6,815,184.

The binding specificity of the antibodies of the present invention can be identified using any technique including those disclosed here, can be determined by immunoprecipitation or by an in vitro binding assay, such as radioimmunoassay (RIA) or enzyme-linked immunoabsorbent assay (ELISA). The binding affinity of a monoclonal antibody or portion thereof can be determined by the Scatchard analysis of Munson et al., Anal. Biochem., 107:220 (1980). Art recognized techniques can also be used to alter or optimize particular binding specificities and/or affinities (see, for example, Carter P J, Nature Reviews Immunology 6: 343-357 (2006)).

In certain embodiments, partial antibody sequences derived from antibodies of the invention may be used for producing structurally and functionally related antibodies. For example, antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L. et al., 1998, Nature 332:323-327; Jones, P. et al., 1986, Nature 321:522-525; Tamura et al., J. Immunol., 2000 Feb. 1; 164(3):1432-41; and Queen, C. et al., 1989, Proc. Natl. Acad. See. U.S.A. 86:10029-10033). Such framework sequences can be obtained from public DNA databases that include germline antibody gene sequences.

Thus, in one embodiment, one or more structural features of the particular anti-flagellin antibodies of the invention are used to create structurally related anti-flagellin antibodies that retain the functional properties of the parent antibodies of the invention, such as binding to the same epitope or overlapping epitopes bound by the anti-flagellin antibodies exemplified herein, as well as competing for antigen-binding with the anti-flagellin antibodies exemplified herein.

In another embodiment, one or more structural features of the particular antibodies of the invention are used to create structurally related anti-flagellin antibodies that retain functional properties of the parent antibodies of the invention, such as (i) neutralizing flagellin; (ii) inhibiting the activity of flagellin; (iii) cross-reacting with a broad spectrum of gram-negative bacteria; (iv) inhibiting bacterial invasion into susceptible epithelial cells; (v) binding to flagellin with an affinity of at least 10⁶ M⁻¹; (vi) reducing the symptoms of an enterobacterial infection or IBD in a subject; (vii) reducing the extent and severity of flagellin-induced tissue injury; (viii) reducing flagellin-stimulated neutrophil infiltration; (ix) decreasing colonic mucosal congestion, erosion and hemorrhagic ulcerations associated with IBD; (x) inhibiting or decreasing the production of mediators (e.g., MDA, IL-1β, TNFα, MIP-1, MIP-2 and IL-8); and (xi) counteracting a reduction in body weight associated with IBD.

Methods known in the art for creating such structural and functional related antibodies include, for example, Marks et al. (Biotechnology (N Y). 1992 July; 10(7):779-83) (monoclonal antibodies diversification by shuffling light chain variable regions, then heavy chain variable regions with fixed CDR3 sequence changes), Jespers et al., (Biotechnology (N Y). 1994 September; 12(9):899-903) (selection of human antibodies from phage display repertoires to a single epitope of an antigen), Sharon et al., (Proc Natl Acad Sci USA. 1986 April; 83(8):2628-31) (site-directed mutagenesis of an invariant amino acid residue at the variable-diversity segments junction of an antibody); Casson et al., (J Immunol. 1995 Dec. 15; 155(12):5647-54) (evolution of loss and change of specificity resulting from random mutagenesis of an antibody heavy chain variable region).

In one embodiment, one or more CDR regions of antibodies of the invention can be combined recombinantly with known human framework regions and CDRs to create additional, recombinantly-engineered, anti-flagellin antibodies of the invention. The heavy and light chain variable framework regions can be derived from the same or different antibody sequences.

It is well known in the art that antibody heavy and light chain CDR3 domains play a particularly important role in the binding specificity/affinity of an antibody for an antigen. See, for example, Brummel et al. (Biochemistry. 1993 Feb. 2; 32(4):1180-7), which showed that binding activity is retained in a wide range of CDR3 mutants for each of the four residues that directly hydrogen bond to the antigen. Only Gly¹⁰² could not be replaced without significant loss of affinity (see also, Hall et al., J. Immunol., 149:1605-1612 (1992); Polymenis et al., J. Immunol., 152:5318-5329 (1994); Jahn et al., Immunobiol., 193:400-419 (1995); Klimka et al., Brit. J. Cancer, 83:252-260 (2000); Beiboer et al., J. Mol. Biol, 296:833-849 (2000); Rader et al., Proc. Natl. Acad. Sci. USA, 95:8910-8915 (1998); Barbas et al., J. Am. Chem. Soc., 116:2161-2162 (1994); Ditzel et al., J. Immunol., 157:739-749 (1996)). Accordingly, in certain embodiments, antibodies can be prepared to include the heavy and/or light chain CDR3s of the antibodies of the present invention (e.g., SEQ ID NOs:9 and 15). The antibodies can further include the heavy and/or light chain CDR2s of the antibodies of the present invention (e.g., SEQ ID NOs:7 and 13). The antibodies can further include the heavy and/or light chain CDR1s of the antibodies of the present invention (e.g., SEQ ID NOs:5 and 11).

The CDR1, 2, and/or 3 regions of the engineered antibodies described above can comprise the exact amino acid sequences as those disclosed herein (e.g., CDRs of monoclonal antibody 743 (“mAb 743”), set forth in SEQ ID NOs: 5, 7, 9, 11, 13 and 15. However, the ordinarily skilled artisan will appreciate that some deviation from the exact CDR sequences may be possible while still retaining the ability of the antibody to bind flagellin effectively (e.g., conservative amino acid substitutions). Accordingly, in another embodiment, the engineered antibody may be composed of one or more CDRs that are, for example, 90%, 95%, 98%, 99% or 99.5% identical to one or more CDRs of mAb 743.

In another embodiment, one or more residues of a CDR may be altered to modify binding to achieve a more favored on-rate of binding. Using this strategy, an antibody having ultra high binding affinity of, for example, 10¹⁰ M⁻¹ or more, can be achieved. Affinity maturation techniques, well known in the art and those described herein, can be used to alter the CDR region(s) followed by screening of the resultant binding molecules for the desired change in binding. Accordingly, as CDR(s) are altered, changes in binding affinity as well as immunogenicity can be monitored and scored such that an antibody optimized for the best combined binding and low immunogenicity are achieved.

In addition to, or instead of, modifications within the CDRs, modifications can also be made within one or more of the framework regions, FR1, FR2, FR3 and FR4, of the heavy and/or the light chain variable regions of an antibody, so long as these modifications do not eliminate the binding affinity of the antibody.

In another embodiment, it may be desirable to modify the antibody of the invention with respect to effector function, so as to enhance the effectiveness of the antibody in treating an infection in a subject, for example. For example cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al. Cancer Research 53:2560-2565 (1993). Alternatively, an antibody can be engineered which has dual Fc regions and may thereby have enhanced complement lysis and ADCC capabilities. See Stevenson et al. Anti-Cancer Drug Design 3:219-230 (1989).

Also encompassed by the present invention are bispecific antibodies and immunoconjugates, as discussed below.

(ii) Bispecific Antibodies

The present invention also provides bispecific antibodies which include at least one anti-flagellin antibody of the invention linked to one or more antibodies which bind to a second target (such as an immune cell (e.g., an Fc receptor on an immune cell) or a different epitope on flagellin). Bispecific antibodies can be prepared as full length antibodies or antibody fragments (e.g. F(ab′)₂ bispecific antibodies). In a particular embodiment, the invention provides mAb 743 linked to one more antibodies which binds to a different epitope on flagellin.

Methods for making bispecific antibodies are well known in the art. For example, production of full length bispecific antibodies can be based on the coexpression of two immunoglobulin heavy chain-light chain pairs, where the two chains have different specificities (see, e.g., Millstein et al., Nature, 305:537-539 (1983)). Further details of generating bispecific antibodies can be found, for example, in Suresh et al., Methods in Enzymology, 121:210 (1986) and in Brennan et al., Science, 229: 81 (1985), which describes a chemical linkage process for making bispecific antibodies. Various techniques for making and isolating bispecific antibody fragments directly from recombinant cell culture have also been described. For example, bispecific antibodies have been produced using leucine zippers (see, e.g., Kostelny et al., J. Immunol., 148(5):1547-1553 (1992)). Another strategy for making bispecific antibody fragments by the use of single-chain Fv (sFv) dimers has also been reported (see, e.g., Gruber et al., J. Immunol., 152:5368 (1994)).

(iii) Immunoconjugates

In another aspect, the present invention provides immunoconjugates that bind to flagellin and target therapeutic agents (e.g., a toxin) to particular classes of bacteria. Immunoconjugates can be formed by conjugating (e.g., chemically linking or recombinantly expressing) antibodies of the invention to suitable therapeutic agents. In a particular embodiment, the invention provides mAb 743 linked to a therapeutic agent. Suitable agents include, for example, a cytotoxic agent, a toxin (e.g. an enzymatically active toxin of bacterial, fungal, plant or animal origin, or fragments thereof), and/or a radioactive isotope (i.e., a radioconjugate). Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. A variety of radionuclides are available for the production of radioconjugated anti-flagellin antibodies. Examples include ²¹²Bi, ¹³¹I, ¹³¹In, ⁹⁰Y and ¹⁸⁶Re.

Immunoconjugates of the invention can be made using a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody (see, e.g., WO94/11026).

### III. Methods for Screening Anti-Flagellin Antibodies

Subsequent to producing antibodies that bind to flagellin, the antibodies can be screened and selected for various properties, such as (i) their effect on bacterial invasion into susceptible epithelial cells, (ii) inhibition of flagellin-stimulated NO or IL-8 production from epithelial cells, (iii) bacterial opsonophagocytosis, (iv) macrophage ingestion of bacteria, (v) superoxide production, (vi) ability to neutralize flagellin, (vii) ability to inhibit the activity of flagellin, (viii) cross-reactivity with a broad spectrum of gram-negative bacteria, (ix) ability to inhibit bacterial invasion into susceptible epithelial cells, (x) ability to bind to flagellin with an affinity of at least 10⁶ M⁻¹, (xi) capability of reducing the symptoms of an enterobacterial infection or IBD in a subject, (xii) capability of reducing the extent and severity of flagellin-induced tissue injury, (xiii) capability of reducing flagellin-stimulated neutrophil infiltration; (xiv) capability of decreasing colonic mucosal congestion, erosion and hemorrhagic ulcerations associated with IBD; (xv) capability of inhibiting or decreasing the production of mediators (e.g., MDA, IL-1β, TNFα, MIP-1, MIP-2 and IL-8); and (xvi) capability of counteracting a reduction in body weight associated with IBD, using a variety of assays that are well known in the art. Assays for screening for such properties include the assays exemplified and described herein, as well as those well known in the art, such as binding to immobilized recombinant or bacterial flagellin on ELISA, binding to recombinant or bacterial extracts on SDS-PAGE, affinity binding determinations to purified antigens by BIACore analysis.

Antibodies or antigen binding portions thereof that bind to the same or overlapping epitopes as one or more antibodies of the present invention can also be identified using standard techniques known in the art and described herein. For example, in order to screen for antibodies which bind to the same or an overlapping epitope on flagellin bound by an antibody of interest, a cross-blocking assay, such as that described in Antibodies, A Laboratory Manual, Cold Spring Harbor Laboratory, Ed Harlow and David Lane (1988), can be performed.

### IV. Pharmaceutical Compositions

In another aspect, the present invention provides compositions, e.g., a pharmaceutical composition, containing one or a combination of antibodies of the invention thereof, of the present invention, formulated together with a pharmaceutically acceptable carrier. In one embodiment, the compositions include a combination of multiple (e.g., two or more) isolated antibodies of the invention, which bind different epitopes on flagellin.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., antibody, bispecific and multispecific molecule, may be coated in a material to protect the compound from the action of acids and other natural conditions that may inactivate the compound.

A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

Pharmaceutical compositions of the invention can be administered alone or in combination therapy, i.e., combined with other agents. For example, the combination therapy can include a composition of the present invention with at least one or more additional therapeutic agents, such as chemotherapeutic agents. The pharmaceutical compositions of the invention can also be administered in conjunction with radiation therapy.

Pharmaceutical compositions of the invention can administered by a variety of methods known in the art. As will be appreciated by the skilled artisan, the route and/or mode of administration will vary depending upon the desired results. The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

To administer a compound of the invention by certain routes of administration, it may be necessary to coat the compound with, or co-administer the compound with, a material to prevent its inactivation. For example, the compound may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al. (1984) J. Neuroimmunol. 7:27).

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. For example, the human antibodies of the invention may be administered once or twice weekly by subcutaneous injection or once or twice monthly by subcutaneous injection.

It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Therapeutic compositions of the present invention include those suitable for oral, nasal, topical (including buccal and sublingual), rectal, vaginal and/or parenteral administration. The formulations may conveniently be presented in unit dosage form and may be prepared by any methods known in the art of pharmacy. The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.001 percent to about ninety percent of active ingredient, preferably from about 0.005 percent to about 70 percent, most preferably from about 0.01 percent to about 30 percent.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

When the antibodies of the present invention are administered as pharmaceuticals, to humans and animals, they can be given alone or as a pharmaceutical composition containing, for example, 0.001 to 90% (more preferably, 0.005 to 70%, such as 0.01 to 30%) of active ingredient in combination with a pharmaceutically acceptable carrier.

Regardless of the route of administration selected, antibodies of the present invention and/or the pharmaceutical compositions thereof, are formulated into pharmaceutically acceptable dosage forms by conventional methods known to those of skill in the art.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved. In general, a suitable daily dose of a composition of the invention will be that amount of the compound which is the lowest dose effective to produce a therapeutic effect. Such an effective dose will generally depend upon the factors described above. It is preferred that administration be intravenous, intramuscular, intraperitoneal, or subcutaneous, preferably administered proximal to the site of the target. If desired, the effective daily dose of a therapeutic composition may be administered as two, three, four, five, six or more sub-doses administered separately at appropriate intervals throughout the day, optionally, in unit dosage forms. While it is possible for a compound of the present invention to be administered alone, it is preferable to administer the compound as a pharmaceutical formulation (composition).

Therapeutic compositions can be administered with medical devices known in the art. For example, in a preferred embodiment, a therapeutic composition of the invention can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medications through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art.

In certain embodiments, antibodies of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134), different species of which may comprise the formulations of the inventions, as well as components of the invented molecules; p120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273.

### V. Methods of Using Anti-Flagellin Antibodies

The present invention provides methods of using antibodies that bind to and neutralize bacterial flagellin in a variety of therapeutic and diagnostic applications.

Suitable diseases that can be treated and/or diagnosed using the antibodies provided herein include, for example, IBD, Ulcerative Colitis and Crohn's Disease, as well as infectious diseases, including, but not limited to, gram negative bacterial infections (e.g., enterobacterial infections), sepsis, septic shock, Anthrax (by the bacterium Bacillus anthracis), Bacterial Meningitis (caused by a variety of bacteria, including, but not limited to, Neisseria meningitides, Streptococcus pneumoniae, Listeria monocytogenes, Pseudomonas aeruginosa, Staphylococcus aureus, Streptococcus agalactiae and Haemophilus influenzae), Botulism (caused by bacterium Clostridium botulinum), Brucellosis (caused by bacteria of the genus Brucella), Campylobacteriosis (caused by bacteria of the genus Campylobacter), Cat Scratch Disease (caused by Bartonella henselae and Bartonella clarridgeiae), Cholera (caused by the bacterium Vibrio cholerae), Diphtheria (caused by Corynebacterium diphtheriae), Epidemic Typhus (causative organism is Rickettsia prowazekii), Impetigo (caused by several bacteria, including, Staphylococcus aureus and Streptococcus pyogenes), Legionellosis (caused by bacteria belonging to the genus Legionella), Leprosy (Hansen's Disease) (caused by the bacterium Mycobacterium leprae), Leptospirosis (caused by spirochaetes of the genus Leptospira), Listeriosis (caused by the bacterium Listeria monocytogenes), Lyme Disease (caused by spirochete bacteria from the genus Borrelia), Melioidosis (caused by the bacterium Burkholderia pseudomallei), MRSA infection (caused by Staphylococcus aureus), Nocardiosis (bacterium of the genus Nocardia, most commonly Nocardia asteroides or Nocardia brasiliensis), Pertussis (Whooping Cough) (caused by the bacterium Bordetella pertussis), Plague (caused by the enterobacteria Yersinia pestis), Pneumococcal pneumonia (caused by a variety of bacteria, including, but not limited to, Streptococcus pneumoniae, Staphylococcus aureus, Haemophilus influenzae, Klebsiella pneumoniae, Escherichia coli, Pseudomonas aeruginosa, Moraxella catarrhalis, Chlamydophila pneumoniae, Mycoplasma pneumoniae, and Legionella pneumophila), Psittacosis (caused by a bacterium called Chlamydophila psittaci), Q fever (caused by infection with Coxiella burnetii), Rocky Mountain Spotted Fever (RMSF) (by Rickettsia rickettsii), Salmonellosis (caused by bacteria of the genus Salmonella), Scarlet Fever, Shigellosis (caused by bacteria of the genus Shigella), Syphilis (caused by Treponema pallidum), Tetanus (Clostridium tetani), Trachoma, Tuberculosis (caused by mycobacteria, mainly Mycobacterium tuberculosis), Tularemia (by the bacterium Francisella tularensis), Typhoid Fever (caused by the bacterium Salmonella typhi), and Urinary Tract Infections (caused by bacteria such as Escherichia coli, Staphylococcus saprophyticus, Proteus mirabilis, Klebsiella pneumoniae, Enterobacter spp., Pseudomonas and Enterococcus).

Antibodies of the present invention are particularly useful for treating enterobacterial infections, and can be selected for broad reactivity with multiple entobacterial strains, such Alishewanella, Alterococcus, Aquamonas, Aranicola, Arsenophonus, Azotivirga, Blochmannia, Brenneria, Buchnera, Budvicia, Buttiauxella, Cedecea, Citrobacter, Dickeya, Edwardsiella, Enterobacter, Erwinia, Escherichia, Ewingella, Grimontella, Hafnia, Klebsiella, Kluyvera, Leclercia, Leminorella, Moellerella, Morganella, Obesumbacterium, Pantoea, Pectobacterium, Candidatus Phlomobacter, Photorhabdus, Plesiomonas, Pragia Proteus, Providencia, Rahnella, Raoultella, Salmonella, Samsonia, Serratia, Shigella, Sodalis, Tatumella, Trabulsiella, Wigglesworthia, Xenorhabdus, Yersinia and Yokenella.

The antibodies can be administered alone or with other therapeutic agents, which act in conjunction with or synergistically with the antibodies, to treat diseases. Such therapeutic agents include, for example, toxins, chemotherapeutic agents, small molecules and radiation

Also within the scope of the present invention are kits comprising antibodies (or immunoconjugates or bispecific antibodies) of the invention which optionally include instructions for use in treating a disease associated with flagellin. The kits may include a label indicating the intended use of the contents of the kit. The term label includes any writing, marketing materials or recorded material supplied on or with the kit, or which otherwise accompanies the kit.

Other embodiments of the present invention are described in the following Examples.

The present invention is further illustrated by the following examples which should not be construed as further limiting. The contents of Sequence Listing, figures and all references, patents and published patent applications cited throughout this application are expressly incorporated herein by reference.

### EXAMPLES

### Example 1

### Generation of Anti-Flagellin Antibodies

### Antigen Construction

The gene fragment corresponding to amino acids 1-156 of the flagellin gene of Salmonella muenchen was used as an antigen (Genbank Accession No. GI: 47233)

(SEQ ID NO: 25):
aaggaaaagatcatggcacaagtcattaatacaaacagcctgtcgctgtt
gacccagaataacctgaacaaatcccagtccgctctgggcaccgctatcg
agcgtctgtcttccggtctgcgtatcaacagcgcgaaagacgatgcggca
ggtcaggcgattgctaaccgtttcaccgcgaacatcaaaggtctgactca
ggcttcccgtaacgctaacgacggtatctccattgcgcagaccactgaag
gcgcgctgaacgaaatcaacaacaacctgcagcgtgtgcgtgaactggcg
gttcagtctgctaacggtactaactcccagtctgaccttgactctatcca
ggctgaaatcacccagcgtctgaacgaaatcgaccgtgtatccggtcaga
ctcagttcaacggcgtgaaagtcctggcgcaggacaacaccctgaccatc
caggttggtgccaacgac

The antigen was prepared by expression of a cDNA clone obtained by PCR amplification of DNA from S. muenchen using a sense primer designated 1S (5′-CGCGGATCCCAATGGCACAAGTCATTAATACAAACA) (SEQ ID NO:17) and an antisense primer designated 468A (5′-TCCGCTCGAGTTAAATAGTTTCACCGTCGTTGGCACC) (SEQ ID NO:18). Underlined nucleotides represent adaptor sequences added to the ends of primers to maintain proper reading frame and facilitate cloning (BamHI recognition sites on sense primers and XhoI sites on antisense primers). The template DNA for PCR was plasmid CL402, a clone of pBR322 containing a 3.8-kb EcoRI fragment of S. muenchen chromosomal DNA that harbors the 1.5-kb flagellin gene. PCR-generated flagellin DNA were digested with BamHI plus XhoI, gel purified, and subcloned into the BamHI/XhoI sites at the 3′ end of the His tag in expression vector pET 30C (Novagen, San Diego, Calif.). The correct reading frame and integrity of subcloned DNA was verified by DNA sequence analysis. The recombinant plasmids were then introduced into Escherichia coli BL21 (DE3) (Novagen) by transformation and selected in the presence of kanamycin (50 ug/ml).

### Expression and Purification of Recombinant Antigen

A single colony of E. coli containing the recombinant plasmid was grown at 37° C. in Luria broth containing 50 ug/ml kanamycin to an A600 of 0.5 and then induced for 3 h with 0.5 mM isopropyl-1-thio-b-D-galactopyranoside. Following induction, bacteria were harvested and washed with phosphate-buffered saline (PBS, pH 7.2). Cell-free lysates were prepared in 6 M guanidine chloride containing 5 mM imidazole and 0.1% Nonidet P-40 (binding buffer). After removing the insoluble material by centrifugation, the lysate was applied to a nickel-nitrilotriacetic acid-agarose (Qiagen, Valencia, Calif.) column, washed extensively with binding buffer, and then eluted with binding buffer containing 200 mM imidazole. The purified proteins were extensively dialyzed against PBS, and protein concentrations were determined by the Bradford method. The final proteins were analyzed by 10% SDS-PAGE and visualized with Coomassie Blue staining to assess protein purity, integrity, and concentration.

### Immunization

Female BALB/c mice of 12 week old were immunized with 50 ug of fusion protein in complete adjuvant. On day 14, 28 and 42 mice were boosted with 50 ug of protein in incomplete adjuvant. Three days after final boost, spleen cells will be prepared for fusion with SP2/O myeloma cells (ATCC). Antibody titer in serum was measured by ELISA and using recombinant S. muenchen flagellin as antigen.

### Preparation of Monoclonal Antibodies

Anti-flagellin monoclonal antibodies were produced using previously described methods (Harlow and Lane, Antibodies, CSH laboratories). Spleens from immunized mice were broken apart with sterile forceps and passed through a sterile stainless-steel strainer by pressing the spleen tissue with the glass plunger. Splenocytes were collected, washed once in serum-free DMEM medium and then fused with SP2/O myeloma cells using sterile PEG (polyethylene glycol) solution. After fusion cells were seeded in 96-well microtiter plates and selected against HAT medium. Hybridoma culture supernatants were screened by ELISA using 96 well plates coated with recombinant flagellin and amplified the positive ones and tested for Ig-subclass (Southern Biotechnology Associates). The candidate hybridoma lines producing IgG subclass were selected and cloned by limited dilution.

### Purification of Monoclonal Antibodies

The murine monoclonal antibody to flagellin was purified using standard immunology techniques. In brief, hybridoma cells were grown in roller bottles for 14 days in BD medium and tissue culture supernatants wee collected. Antibodies were further purified by passing over a 10 ml protein G-sepharose affinity column, washed extensively with PBS, and eluted with 0.1 M glycine (pH 2.5). After dialysis of the eluate against PBS, antibodies titers were tested by ELISA and tested the purity by SDS-polyacrylamide gels.

### Example 2

### Hybridoma Sequencing

mRNA Preparation

mRNA was extracted from 3×10⁶ thawed hybridoma cells expressing murine mAb 743 using Norgen's Cytoplasmic & Nuclear RNA Purification Kit using the standard procedure. Cells were thawed at room temperature and centrifuged briefly in a microcentrifuge for 10 minutes. Approximately 0.2 ml of lysis buffer containing 2 ul beta-mercaptoethanol was added to the pellet and the mixture transferred to a 1.5 ml eppendorf tube. The tube was vortexed for 15 secs and centrifuged for 3 minutes at room temperature and transferred to a clean 1.5 ml tube. Binding solution (0.2 ml containing beta-mercaptoethanol) was added to supernatant, mixed by vortexing for 10 secs, followed by addition of 160 μl of 100% ethanol and vortexed for 10 secs. The extracted RNA was purified on a spin column according to kit directions.

The light and heavy chains were prepared using distinct mRNA samples in separate reactions. The contaminating light chain variable region pseudogene mRNA produced by the myeloma cells was digested RNase H (proprietary).

Following digestion 5′ RACE was performed using an Ambion FirstChoice RLM-RACE kit according to manufacturer's instruction.

### RT-PCR

Reverse transcription was performed using random decamer primers provided in the Ambion kit. The product cDNA was amplified using outer and inner PCR reactions. Thermocycler was programmed 3 minutes at 94° C. followed by 35 cycles 94° C. for 30 seconds; 60° C. for 30 secs; 72° C. for 30 seconds; followed by 7 min at 72° C. Each PCR reaction used one adaptor-specific primer and one immunoglobulin constant region-specific primer (proprietary). The PCR products (approximate size of 500 bp) were extracted from a 2% agarose gel and purified using Qiaquick gel extraction kit (Qiagen cat #28704).

### Cloning

Purified PCR products of the correct size for the heavy and light chain variable regions were ligated into Invitrogen's TOPO TA cloning vectors and transformed into TOP10 cells according to manufacturer's instructions. The clones were screened by PCR (using the previously described reaction conditions) to find those with the correct-sized insert (approximately 650 bp). Light chain clones containing the correct-sized insert, were grown in Luria broth containing competent cells (Invitrogen OneShot Chemically Competent E. Coli.), purified using a MiniPrep kit (Invitrogen cat # K2100-11) and cycle sequenced (Dartmouth MicroSequencing Facility, Lebanon, N.H.). Heavy chain clones which contained the correct-sized insert, were similarly grown and purified and cycle sequenced.

### Sequences

The sequences were aligned using Sequencher software (Gene Codes Corporation). The results are as follows:

mAb 743 light chain consensus nucleotide sequence:

(SEQ ID NO: 4)

ATTAGCCAGGAACAAAAATTCAAAGACAAAATGGATTTTCAGGTGCAGAT
TTTCAGCTTCCTGCTAATCAGTTTCTCAGTCATAATGTCCAGAGGACAAA
TTGTTCTCACCCAGTCTCCAGCAATCATGTCTGTATCTCCGGGGGAGAAA
GTCACCTTGACCTGCAATGCCAGCTCAAGTGTAAGTTCCAGCTACTTATA
CTGGTATCGGCAGAAACCAGGATCTTCCCCCAACCTCTGGATTTATAGCA
CATCCAACCTGGCTTCTGGAGTCCCTGCTCGCTTCAGTGGCAGTGGGTCT
GGGACCTCTTACTCTCTCACAATCAGCAGCATGGAGGCTGAAGATGTTGC
CTCTTATTTCTGCCATCAGTGGAGTAGTTACCCGCTCACGTTCGGTGCTG
GGACCAAGCTGGAGCTGAAG
mAb 743 light chain amino acid sequence:

(SEQ ID NO: 2)

ISQEQKFKDKMDFQVQIFSFLLISFSVIMSRGQIVLTQSPAIMSVSPGEK
VTLTCNASSSVSSSYLYWYRQKPGSSPNLWIYSTSNLASGVPARFSGSGS
GTSYSLTISSMEAEDVASYFCHQWSSYPLTFGAGTKLELK
mAb 743 light chain CDR1 amino acid sequence:

(SEQ ID NO: 11)

NASSSVSSSYLY
mAb 743 light chain CDR2 amino acid sequence:

(SEQ ID NO: 13)

STSNLAS
mAb 743 light chain CDR3 amino acid sequence:

(SEQ ID NO: 15)

HQWSSYPLT
mAb 743 heavy chain consensus nucleotide sequence:

(SEQ ID NO: 3)

GCGGCCGCGAATTCGCCCTTCGCGGATCCGAACACTGCGTTTGCTGGCTT
TGATGAAAATAAGGTCACTGTTCTCACTATAGTTACTGAGCACACAGACC
TCACCATGGGATGGAGCTCTATCATCCTCTTCTTGTTAGCAACAGCTACA
GTGTCCACTCCCAGGTCCAACTGCAGCAGCCTGGGGCTGAGCTGGTGAG
GCCTGGAGCTTCAGTGAAGCTGTCCTGCAAGGCTTCTGGCTACTCCTTCA
CCAGCGACTGGATGAACTGGGTGAAACAGAGGCCTGGACAGGGCCTTGAG
TGGATTGGCATAATTCATCCTTCCGACAGTGAAACTAAAATAAATCAATA
TTTCAAGGACAAGGCCACATTGACTATAGACAAATCCTCCAGCACAGCCT
ACATGCAACTCAGCAGCCCGACATCTGAGGACTCTGCGGTCTATTACTGT
GTTAGATGGGTGGGCTACCACGCTCAGGTCTGGGGCCAAGGCACCACTCT
CACCGTC
mAb 743 heavy chain amino acid sequence:

(SEQ ID NO: 1)

AAANSPFADPNTAFAGFDENKVTVLTIVTEHTDLTMGWSSIILFLLATAT
GVHSQVQLQQPGAELVRPGASVKLSCKASGYSFTSDWMNWVKQRPGQGLE
WIGIIHPSDSETKINQYFKDKATLTIDKSSSTAYMQLSSPTSEDSAVYYC
VRWVGYHAQVWGQGTTLTV
mAb 743 heavy chain CDR1 amino acid sequence:

(SEQ ID NO: 5)

GYSFTSDWMN
mAb 743 heavy chain CDR2 amino acid sequence:

(SEQ ID NO: 7)

IIHPSDSETKINQYFKD
mAb 743 heavy chain CDR3 amino acid sequence:

(SEQ ID NO: 9)

WVGYHAQV

### Example 3

### Generation of Humanized Antibodies

Antibodies of the invention can also be humanized using a variety of known techniques known in the art, such as those taught in U.S. Pat. No. 5,225,539 to Winter, and U.S. Pat. Nos. 5,530,101; 5,585,089; 5,693,762 and 6,180,370 to Queen et al., the substance of which is incorporated herein by reference. Additionally, the antibodies of the invention can be humanized using composite human antibody technologies, as described below.

A. Design of Humanized Heavy and Light Chains

The sequences of mAb 743 (or other anti-flagellin antibodies) heavy and light chain variable regions can be analyzed to identify CDRs, unusual amino acids and residues critical to binding as follows.

First, protein models of the murine antibody variable regions can be generated using existing antibody structures as templates. Structural information from the protein model can then be used to identify and compare residues critical for antibody conformation and binding with structurally equivalent residues from existing antibody structures and sequence databases. These amino acids can then be candidates for inclusion in one or more variants of the final humanized sequences.

Segments of monoclonal antibody heavy and light chain variable region amino acid sequences can then be compared with corresponding segments of human variable region sequences in order to identify potential heavy and light chain human sequences for possible inclusion in the humanized sequences.

A series of at least ten of each humanized heavy and light chain variable regions can then be designed entirely from segments of human variable region sequences. Alternative variants will differ in the inclusion of residues which might be critical to restoration of the original monoclonal antibody binding efficiency with the objective that the number of alterations in the frameworks needed to restore binding efficiency will be kept to the minimum and generation of T-cell epitopes avoided. Potential T cell epitopes as determined by in silico methods can be considered in the selection of alternative variants.

B. Construction of Humanized Heavy and Light Chains

Humanized variable regions can be constructed by PCR-ligation of long synthetic oligonucleotides. The initial heavy and light chain variable region genes can be used as templates for construction of additional sequences by mutagenesis using overlapping PCR with mutagenic oligonucleotide primers. Restriction enzyme sites can then be engineered upstream and downstream of each of the variable heavy and light chains for cloning into the appropriate expression vector (e.g., an Antitope expression vector). The entire DNA sequence can be confirmed to be correct for each modified variable region cassette.

C. Construction of Expression Plasmids Encoding Humanized Antibodies with Human Constant Regions

At least ten humanized variable regions can be transferred into mammalian expression vectors as follows. First, the DNA sequences for each variable region can be inserted into mammalian expression vectors between an upstream cytomegalovirus immediate/early promoter/enhancer (CMV-ie) plus the immunoglobulin signal sequence and a downstream the immunoglobulin constant region. The heavy chain vector includes a genomic human IgG constant region of choice (IgG1, or IgG4) and the dhfr gene for selection in mammalian cells. The light chain vector includes the genomic human κ constant region.

DNA samples can then be prepared for transfection into mammalian cells. The humanized antibody heavy and light chain-encoding plasmids can be co-transfected into mammalian cells by electroporation.

D. Generation of Humanized Antibody-Producing Cell Lines and Selection of Lead Humanized Antibody

Individual heavy and light chain plasmids can be paired in order to produce a final series of antibodies combining variant humanized variable region sequences. These combinations may also include chimeric heavy and light chains in order to determine the effects of individual modified humanized chains on binding efficiency.

Heavy and light chain plasmid DNA pairs can then be transfected into mammalian cells by electroporation, stable cell lines can be selected and tested for antibody production. Cell lines producing humanized antibodies comprising combinations of heavy and light chains can then be expanded and antibody samples (typically 100 ug) purified.

Antibodies can then be tested in a binding assay in order to determine the humanized best antibody lead. The entire coding sequence of the lead humanized antibody can then be subcloned into an appropriate mammalian expression vector.

### Example 4

### Generation of Human Antibodies

The present invention also contemplates the generation of fully human antibodies. Such antibodies can be generated using a variety of techniques known in the art, such as those described above, including, but, not limited to the techniques described by Hoet et al (2005) Nature Biotechnology 23, 344-348, Lonberg, et al. (1994) Nature 368(6474): 856-859; Lonberg, N. et al. (1994), supra; reviewed in Lonberg, N. (1994) Handbook of Experimental Pharmacology 113:49-101; Lonberg, N. and Huszar, D. (1995) Intern. Rev. Immunol. 13: 65-93, and Harding, F. and Lonberg, N. (1995) Ann. N.Y. Acad. Sci. 764:536-546. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Publication Nos. WO 92/03918, WO 93/12227, WO 94/25585, WO 97/13852, WO 98/24884 and WO 99/45962, all to Lonberg and Kay; and PCT Publication No. WO 01/14424 to Korman et al., the substance of which is incorporated herein by reference.

### Example 5

### Binding Studies

The binding of mAb 743 to Pseudomonas aeruginosa type A flagellin and type B flagellin was assessed via SDS-PAGE and Western Blotting. Bacterial cells grown overnight in Luria Broth were harvested by centrifugation and then stored at −800 C. After thawing, the pellets were suspended in phosphate buffered saline (PBS) and sonicated to break chromosomal DNA. Bacterial lysates were boiled in equal volumes of loading buffer (125 mM Tris-HCl, pH 6.8, 4% SDS, 20% glycerol, and 10% 2-mercaptoethanol), and 20 μg protein loaded per lane on an 8-16% Tris-glycine gradient gel (Novex, San Diego, Calif. The proteins were then transferred to nitrocellulose (Novex) using the Novex Xcell Mini-Gel system. Membranes were then blocked with 1% bovine serum albumin (BSA) in PBS-T (0.05% tween 20) for 1 hour on an orbital shaker. The blocked membranes were then incubated with INO-743 (0.1 ug/ml) in 1% BSA/PBS-T for 1 hour, washed with PBS-T to remove unbound antibodies, and incubated with Rabbit anti-mouse IgG-HRP 1:5000 in 1% BSA/PBS-T for 1 hour. The blots were washed as above and ECL chemiluminescent reagent added to the membrane for 1 minute. The blots were then exposed to X-ray film.

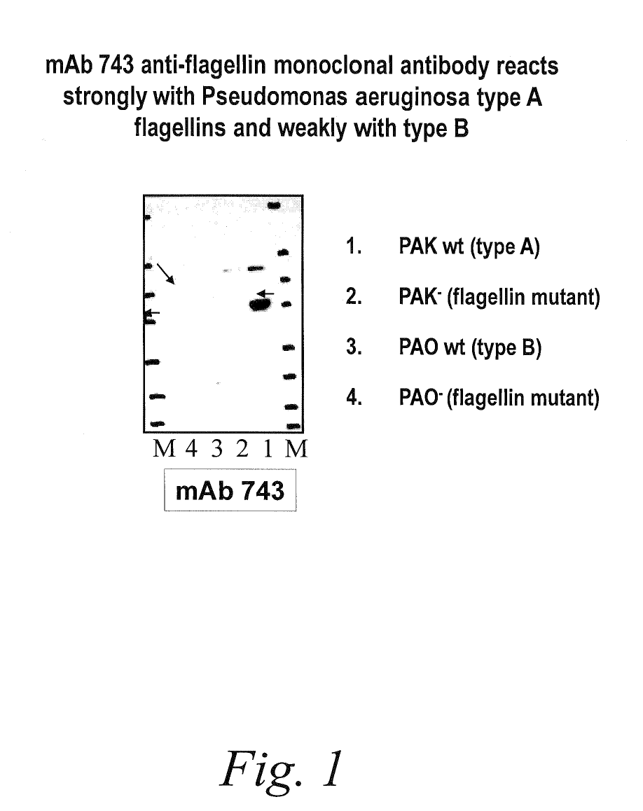

As shown in FIG. 1, mAb 743 reacts strongly with Pseudomonas aeruginosa type A flagellin and binds weakly with Pseudomonas aeruginosa type B flagellin.

### Example 6

### Anti-Flagellin Monoclonal Antibody 743 Cross-Reacts with a Broad Spectrum of Gram-Negative Bacteria

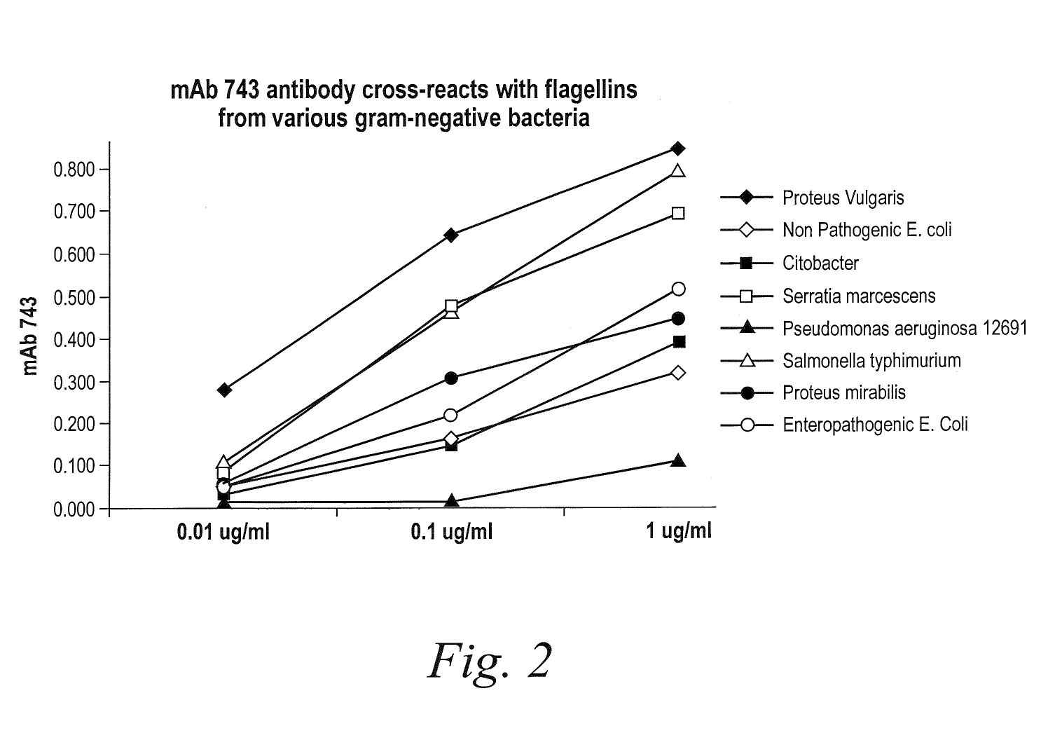

Dose-response binding relative to mAb 743 was assessed towards a panel of different gram-negative bacteria (i.e., Proteus Vulgaris, non-pathogenic E. Coli, Citrobacter, Serratia marcenscens, Pseudomonas aeruginosa, Salmonella typhimurium, Proteus mirabilis, Enteropathogenic E. Coli) in a live bacterial ELISA assay. mAb 743 bound cross-reactively to a broad variety of different bacteria with highest binding observed towards Proteus Vulgaris, Salmonella typhimurium and Serratia marcenscens (FIG. 2).

### Example 7

### Epitope Mapping Studies

The epitope of Salmonella muenchen flagellin bound by mAb 743 was determined by immunoblotting and immunoprecipitation assays. Specifically, fine epitope mapping was performed by assessing the binding of antibodies to various peptides corresponding to various regions of flagellin protein. Recombinant proteins containing amino acids corresponding to 1-40, 1-156, 41-156, 78-156 and 53-505 were expressed in E. coli and purified to homogeneity using Ni affinity columns. Protein amounts equivalent to 1 μg were separated on an 8-16% Tris-glycine gradient gel (Novex, San Diego, Calif.). The proteins were then transferred to nitrocellulose (Novex) using the Novex Xcell Mini-Gel system. Membranes were then blocked with 1% bovine serum albumin (BSA) in PBS-T (0.05% tween 20) for 1 hour on an orbital shaker. The blocked membranes were then incubated with INO-743 (0.1 μg/ml) in 1% BSA/PBS-T for 1 hour, washed with PBS-T to remove unbound antibodies, and incubated with Rabbit anti-mouse IgG-HRP 1:5000 in 1% BSA/PBS-T for 1 hour. The blots were washed as above and ECL chemiluminescent reagent added to the membrane for 1 minute. The blots were then exposed to X-ray film.

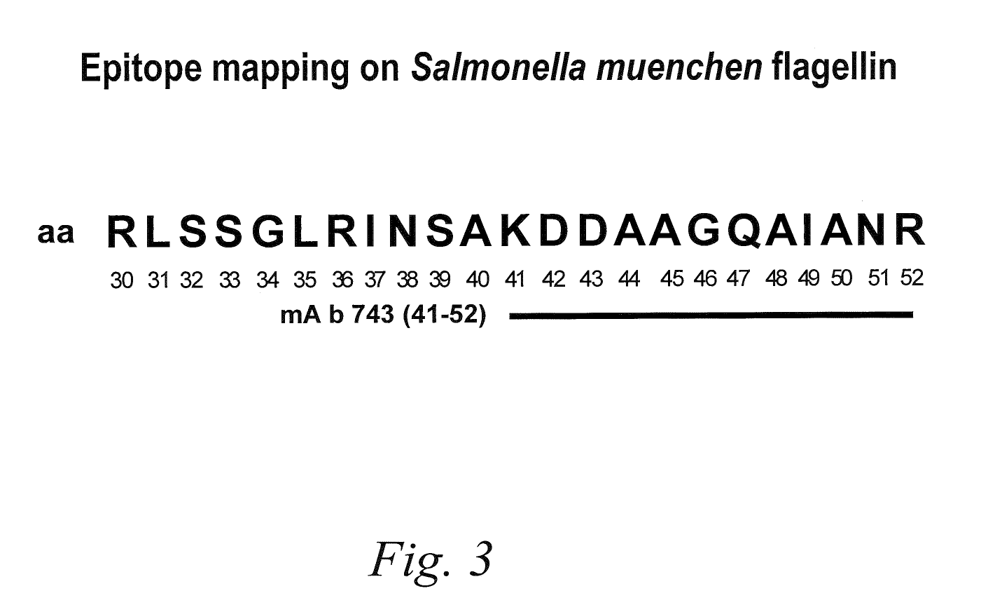

As shown in FIG. 3, mAb 743 mapped to amino acids 41-53 of Salmonella muenchen (SEQ ID NO:23).

### Example 8

### In Vitro Nitric Oxide Production Assay

An in vitro nitric oxide (NO) production assay was used to assess the functional characteristics of murine mAb 743. A non-specific monoclonal antibody and flagellin alone were used as controls. DLD-1 cells (ATCC) were grown at 37° C. in 5% CO2 in Dulbecco's modified Eagle's medium (DMEM, Invitrogen) and were supplemented with 10% FBS, 4 mM glutamine, 1.5 g/liter sodium bicarbonate, 4.5 g/liter glucose, 1 mM sodium pyruvate, and antibiotics. Cells, between passages 5 and 15, were seeded at a density of 50,000 cells/cm2 in 96-well plates and allowed to grow 72-96 hours to confluence before use. Growth medium was changed the day before use. Cells were washed once with DMEM without FBS (but containing antibiotics) before the addition of flagellin proteins. Flagellin to be tested were added to 100 μl growth medium containing 0.5% FBS and 100 U/ml IFN-γ in each well. To neutralize NO production activity, the recombinant flagellin was incubated with antibodies for 1 hour at room temperature and then added to DLD-1 cells in 96 well plate. After 20 hours of incubation at 370 C, the culture medium was removed and tested for NO2-/NO3-concentration by Greiss assay (Salzman et al. Am J Physiol. 268, 361-73 (1995)).

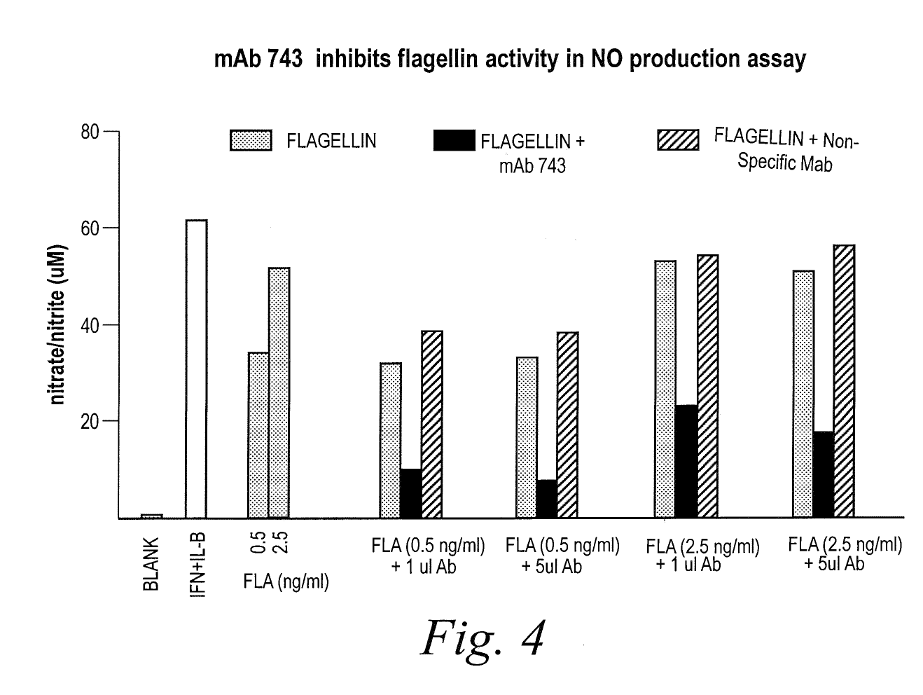

As depicted in FIG. 4, mAb 743 inhibited flagellin activity in the NO production assay.

### Example 9

### Additional In Vitro Functional Assays

A number of in-vitro functional assays can be performed to assess the functional characteristics of the anti-flagellin antibody of the present invention (e.g., murine mAb 743). Such assays can include, but are not limited to, assays designed to assess the effect of anti-flagellin antibodies on (1) bacterial invasion into susceptible epithelial cells, (2) inhibition of flagellin-stimulated NO or IL-8 production from epithelial cells, (3) bacterial opsonophagocytosis, (3) macrophage ingestion of bacteria, (4) bacterial “killing” and (5) superoxide production.

### EQUIVALENTS

Those skilled in the art will recognize, or be able to ascertain using no more than routine experimentation, many equivalents of the specific embodiments of the invention described herein. Such equivalents are intended to be encompassed by the following claims. Any combination of the embodiments disclosed in the dependent claims are contemplated to be within the scope of the invention.

### INCORPORATION BY REFERENCE

The contents of all references, patents and published patent applications cited throughout this application, as well as the figures and Sequence Listing, are expressly incorporated herein by reference.

SUMMARY OF SEQUENCE LISTING

| SEQ ID NO: | SEQUENCE |
|---|---|
| 1 | INO-743 VH aa |
| 2 | INO-743 VL aa |
| 3 | INO-743 VH nt |
| 4 | INO-743 VL nt |
| 5 | INO-743 VH aa CDR1 |
| 6 | INO-743 VH nt CDR1 |
| 7 | INO-743 VH aa CDR2 |
| 8 | INO-743 VH nt CDR2 |
| 9 | INO-743 VH aa CDR3 |
| 10 | INO-743 VH nt CDR3 |
| 11 | INO-743 VL aa CDR1 |
| 12 | INO-743 VL nt CDR1 |
| 13 | INO-743 VL aa CDR2 |
| 14 | INO-743 VL nt CDR2 |
| 15 | INO-743 VL aa CDR3 |
| 16 | INO-743 VL nt CDR3 |
| 17 | Sense Primer Designated 1S |
| 18 | Antisense Primer |
| | Designated 468A |
| 19 | Proteus mirabilis |
| | (GI: 1169696) |
| 20 | Pseudomonas aeruginosa (GI: 3386643) |
| 21 | Escherichia coli |
| | (GI: 1655807) |
| 22 | Serratia marcescens |
| | (GI: 514988) |
| 23 | Salmonella muenchen aa (GI: 1333832) |
| 24 | Salmonella typhimurium (GI: 153979) |
| 25 | Salmonella muenchen nt (GI: 47233) |

1. An isolated monoclonal antibody that binds to flagellin, wherein the antibody comprises a heavy chain variable region comprising an amino acid sequence set forth in SEQ ID NO:1.

2. An isolated monoclonal antibody that binds to flagellin, wherein the antibody comprises a light chain variable region comprising an amino acid sequence set forth in SEQ ID NO:2.

3. An isolated monoclonal antibody that binds to flagellin, wherein the antibody comprises a heavy and light chain variable region comprising the amino acid sequences set forth in SEQ ID NOs:1 and 2, respectively.

4. (canceled)

5. (canceled)

6. (canceled)

7. (canceled)

8. An isolated monoclonal antibody that binds to flagellin and comprises heavy and light chain variable region CDR1, CDR2, and CDR3 sequences, wherein:
the heavy chain variable region CDR1 comprises SEQ ID NO:5;
the heavy chain variable region CDR2 comprises SEQ ID NO:6;
the heavy chain variable region CDR3 comprises SEQ ID NO:7;
the light chain variable region CDR1 comprises SEQ ID NO:8;
the light chain variable region CDR2 comprises SEQ ID NO:9; and
the light chain variable region CDR3 comprises SEQ ID NO:10.

9. An isolated antibody that binds to an epitope on flagellin recognized by the antibody of claim 3 or 8.

10. An isolated antibody that competes for binding to flagellin with the antibody of claim 3 or 8.

11. An isolated antibody that specifically binds to an epitope located between amino acids 41-53 of flagellin from Salmonella (SEQ ID NO:17).

12. The antibody of any one of claims 3, 8, or 11, wherein the antibody is selected from the group consisting of a human antibody, a humanized antibody, a chimeric antibody, and a murine antibody.

13. The antibody of any one of claims 3, 8, or 11, wherein the antibody is selected from the group consisting of a Fab, Fab′2, ScFv, SMIP, affibody, avimer, nanobody, and a domain antibody.

14. The antibody of any one of claims 3, 8 or 11, wherein the antibody isotype is selected from the group consisting of an IgG1, an IgG2, an IgG3, an IgG4, an IgM, an IgA1, an IgA2, an IgAsec, an IgD, and an IgE antibody.

15. A hybridoma which produces the antibody of claim 3 or 8.

16. An immunoconjugate comprising the antibody of any one of claims 3 or 8-11, linked to a therapeutic agent.

17. (canceled)

18. A bispecific molecule comprising the antibody of any one of claims 3 or 8-11, linked to a molecule having a binding specificity which is different from said antibody.

19. A composition comprising an antibody of any of claims 3 or 8-11, an immunoconjugate of claim 16, or the bispecific molecule of claim 18, and a pharmaceutically effective carrier.

20. The composition of claim 19, further comprising a therapeutic agent selected from the group consisting of a second antibody and an antibiotic.

21. (canceled)

22. (canceled)

23. An isolated nucleic acid molecule encoding a variable region of an antibody that binds to flagellin, wherein the nucleic acid comprises the nucleotide sequence set forth in SEQ ID NOs:3 or 4.

24. (canceled)

25. (canceled)

26. An expression vector comprising the nucleic acid molecule of claim 23.

27. A kit comprising one or more isolated monoclonal antibodies of any of claims 3 or 8-11, an immunoconjugate of claim 16, or the bispecific molecule of claim 18, and optionally comprising instructions for use in treating or diagnosing a disease or infection associated with flagellin.

28. An isolated cell expressing the antibody of claim 3 or 8.

29. A method of producing the antibody of claim 3 or 8.

30. A method of treating a disease or infection associated with flagellin comprising administering to the subject a therapeutically effective amount of an isolated antibody of claim 3 or 8.

31. A method of treating a gram negative bacterial infection in a subject, comprising administering to the subject a therapeutically effective amount of an isolated antibody of claim 3 or 8.

32. A method of neutralizing enterobacteria comprising contacting the enterobacteria with an isolated antibody of any of claim 3 or 8.

33. The method of claim 31, wherein the gram negative bacterial infection is an enterobacterial infection selected from the group consisting of Anthrax, Bacterial Meningitis, Botulism, Brucellosis, Cat Scratch Disease, Cholera, Diphtheria, Epidemic Typhus, Impetigo, Legionellosis, Leprosy, Leptospirosis, Listeriosis, Lyme Disease, Melioidosis, MRSA infection, Nocardiosis, Pertussis, Plague, Pneumococcal pneumonia, Psittacosis, Q fever, Rocky Mountain Spotted Fever (RMSF), Salmonellosis, Scarlet Fever, Shigellosis, Syphilis, Tetanus, Trachoma, Tuberculosis, Tularemia, Typhoid Fever, sepsis, septic shock and Urinary Tract Infections.

34. (canceled)
